(19) United States
(12) Patent Application Publication
(10) Pub. No.: US 2003/0207340 A1
(43) Pub. Date: Nov. 6, 2003

(54) Sequences encoding human neoplastic marker

(76) Inventors: D. James Morre, West Lafayette, IN (US); Dorothy M. Morre, West Lafayette, IN (US); Pin-ju Chueh, West Lafayette, IN (US)

Correspondence Address: GREENLEE WINNER AND SULLIVAN P C, 5370 MANHATTAN CIRCLE, SUITE 201, BOULDER, CO, 80303, US

(21) Appl. No.: 10/138,195

(22) Filed: May 1, 2002

(51) Int. Cl. C12Q 1/68; G01N 33/574; C07H 21/04; C12N 9/02; C12P 21/02; C12N 1/21; C12N 5/06

(52) U.S. Cl. 435/7.23; 435/6; 435/69.1; 435/189; 435/320.1; 435/252.33; 435/358; 536/23.2

(57) ABSTRACT

The present disclosure provides the nucleotide sequence encoding a cell surface NADH oxidase/protein disulfide-thiol interchange protein (tNOX) characteristic of neoplastic and virus-infected cells. Also provided are recombinant DNA molecules comprising a sequence portion encoding full length or truncated tNOX, recombinant host cell which express full length or truncated tNOX, methods for recombinant production of tNOX or truncated tNOX, and diagnostic methods which employ either nucleotide sequences of the neoplastic cell-specific tNOX or antibodies specific for the rNOX protein.

### CROSS REFERENCE TO RELATED APPLICATIONS

This application claims priority from PCT/US00/30190, filed Nov. 1, 2000, which claims priority from U.S. Provisional Application No. 60/162,644, filed Nov. 1, 1999.

### ACKNOWLEDGMENT OF FEDERAL RESEARCH SUPPORT

This invention was made, at least in part, with funding from the National Institutes of Health Accordingly, the United States Government has certain rights in this invention.

### BACKGROUND OF THE INVENTION

The field of this invention is the area of molecular biology, in particular, as related to the molecular biology of neoplastic and diseased cells, as specifically related to a cell surface marker for neoplastic and certain other diseased cell states.

Because cancer and certain viral, protozoan and parasite infections pose a significant threat to human health and because such infections result in significant economic costs, there is a long-felt need in the art for an effective, economical and technically simple system in which to assay for or model for inhibitors of the aforementioned disease states.

### SUMMARY OF THE INVENTION

An object of the present invention is to provide a recombinant plasma membrane NADH oxidase/thiol interchange protein (termed tNOX herein) and its coding sequence. The full length protein has an amino acid sequence as given in SEQ ID NO: 2, and the truncated tNOX protein has the amino acid sequence given in SEQ ID NO: 2, amino acids 220-610. The full length sequence has a specifically exemplified coding sequence as given in SEQ ID NO: 1, nucleotides 23-1852, and the truncated protein has an amino acid sequence as given at nucleotides 680-1852 of SEQ ID NO: 1. Also within the scope of the present invention are coding sequences which are synonymous with those specifically exemplified sequences. Also contemplated within the present invention are sequences which encode a neoplastic cell surface marker and which coding sequences hybridize under stringent conditions to the specifically exemplified full length or partial sequence. The cell surface tNOX is characteristic of neoplastic conditions and certain viral and other infections (e.g., HIV). The recombinant tNOX protein is useful in preparing antigen for use in generation of monoclonal antibodies or antisera for diagnosis of cancer, other neoplastic conditions, and certain infectious disease states.

Within the scope of the present invention are non-naturally occurring recombinant DNA molecules comprising a portion encoding an NADH oxidase/protein disulfide-thiol interchange polypeptide, said portion consisting essentially of a nucleotide sequence selected from the group consisting of SEQ ID NO: 1, nucleotides 23 to 1852; SEQ ID NO: 1, nucleotides 680 to 1852; and a sequence which hybridizes under stringent conditions to one of the foregoing sequences and wherein said hybridizing sequence encodes a neoplastic marker protein of the cell surface having NADH oxidase/protein disulfide-thiol interchange activity. These recombinant DNA molecules can include sequences where the encoded polypeptide consists essentially of an amino acid sequence of SEQ ID NO: 2, amino acids 1 to 610 or amino acids 220 to 610. The portion encoding the specified polypeptide can further contain a translation termination codon (TGA, TAA or TAG) immediately downstream of nucleotide 1852 of SEQ ID NO: 1. Also provided herein are methods for recombinantly producing a NADH oxidase/protein disulfide-thiol interchange active polypeptide in a host cell (bacterial, yeast, mammalian) using the recombinant DNA molecules provided herein.

The present invention further provides a method for determining neoplasia in a mammal, said method comprising the steps of detecting the presence, in a biological sample from a mammal, of a ribonucleic acid molecule encoding a NADH oxidase/protein disulfide thiol interchange protein associated with neoplastic cells as compared to a ribonucleic acid molecule encoding a NADH oxidase associated with normal cells, wherein the step of detecting is carried out using hybridization under stringent conditions or using a polymerase chain reaction in which a perfect match of primer to template is required, where a hybridization probe or primer consists essentially consists essentially of at least 15 consecutive nucleotides of a nucleotide sequence as given in SEQ ID NO: 1 and correlating the result obtained with said sample in step (a), where the presence of the ribonucleic acid molecule in the biological sample is indicative of the presence of neoplasia. The method encompasses the use of hybridization probes which consist essentially of a nucleotide sequence as given in SEQ ID NO: 1, nucleotides 680-1852, nucleotides 23 to 1852 or a portion thereof where there is a detectable difference in the results obtained with normal cells as compared to neoplastic cells or virus infected cells.

The present invention enables the generation of antibody preparations, especially using recombinant tNOX or truncated tNOX or an antigenic peptide derived in sequence from tNOX, which specifically binds to an antibody selected from the group consisting of a protein characterized by an amino acid sequence as given in SEQ ID NO: 2, amino acids 1-610, a protein characterized by an amino acid sequence as given in SEQ ID NO: 220-610 or a protein characterized by an amino acid sequence as given in SEQ ID NO: 16. These antibody preparations are useful in detecting tNOX in blood or serum from a patient or animal with a neoplastic condition such as cancer, or cells or tissue which are neoplastic or virus infected.

Expressing the tNOX of the present invention in a host cell, for example, a mammalian host cell, results in a faster growth rate of the recombinant host cell and a significant increase in recombinant cell volume.

Northern blot analyses indicate that the described cDNA is expressed in HeLa cells (human cervical carcinoma) and malignant BT-20 human mammary adenocarcinoma cells. The availability of the cDNA makes possible rapid further testing of the specificity of expression in a variety of normal and malignant cells and tissues.

The deduced amino acid sequence of the encoded protein showed homology over part of its length with the deduced amino acid sequence of a cDNA encoding a protein detected by the K1 antibody from an ovarian carcinoma (OVCAR-3) cell line [Chang and Pastan (1994) Int. J. Cancer 57:90-97]. The DNA is probably identical to that isolated by Chang and Pastan although their sequence contains two errors that generated an incorrect reading frame. Based on preliminary studies with OVCAR-3 cells, the MAB 12.1 used in the expression screening does not appear to react selectively with an antigen preferentially expressed by OVCAR-3 cells nor do any of the properties of tNOX parallel those of the K1 antigen of OVCAR-3 cells.

To study the biological function of tNOX, the tNOX cDNA was subcloned into a pcDNA3.1 expression vector with HindIII and BamHI restriction sites. Subsequently, COS cells were transfected with tNOX using calcium phosphate transfection and DMSO shock. tNOX overexpression was evaluated on the basis of enzymatic activity and Western blot analysis. Peptide antibody against tNOX recognized expressed proteins with the molecular weights of 34 and 48 kDa. Growth rates determined by image enhanced light microscopy of the tNOX-transfected cells were 2- to 3-fold greater than with vector alone. The larger cell diameter led to a 4- to 5-fold increase in cell volume. A larger cell surface of the transfected cells was confirmed by electron microscopy. As expected, transfected COS cells were more susceptible to tNOX inhibitors, such as capsaicin and epigallocatechin gallate (EGCg), with the EC₅₀ of growth inhibition being shifted by 1 to 2 orders of magnitude to lower drug concentrations. Thus, tNOX function is in cell enlargement and is believed important in sustaining the uncontrolled growth of cancer cells.

### BRIEF DESCRIPTION OF THE DRAWINGS

FIG. 1 summarizes the results of restriction mapping the tNOX cDNA clones.

FIG. 2 diagrammatically illustrates the intron-exon organization of the gene encoding human tNOX. Closed boxes in the genomic DNA map represent the identified protein-coding exons. The tNOX gene is at the Xq25-26 chromosomal locus. At least nine exons have been identified within the partial genomic information available (Bird, 1999).

FIG. 3 is a hydropathy plot prepared using the deduced amino acid sequence of tNOX and the algorithm of Kyte and Doolittle, 1982. One strongly hydrophobic region extending from amino acids 535-558 of SEQ ID NO: 2 was identified.

FIG. 4 shows the results of Western blot analysis of OVCAR-3 cells using antisera raised in a rabbit which was immunized with recombinant tNOX. Following separation on 12% SDS-PAGE, proteins were electroblotted to nitrocellulose and incubated overnight at 4° C. with 1:250 diluted polyclonal antibody to tNOX. Detection was with alkaline phosphatase-conjugated antibody diluted 1:5000 followed by incubation with NBT-BCIP. All fractions were prepared according Chang and Pastan (1994). Lane 1, Membrane pellet after octylglucoside solubilization. Lane 2, Supernatant after octylglucoside solubilization. Arrows indicate immunoreactive unprocessed tNOX (72 kDa) and processed tNOX (34 kDa). The regions of the gel corresponding to APK1 (29 kD) and mesothelin (40 kD) lack immunoreactive material.

FIGS. 5A-5C show the periodic variation in the rate of oxidation of NADH as a function of time over 100 min, with 5 maxima. FIG. 5A: the enzyme source was a crude preparation from bacterial cells expressing the tNOX cDNA from a HeLa library induced to express the protein by the addition of IPTG. FIG. 5B: The crude preparation was as in FIG. 5A except that the expression of the tNOX cDNA cloned under the regulatory control of the lac promoter was not induced. FIG. 5C: The crude preparation was as in FIG. 5A except that the activities were measured as a function of time. The solid curve shows oxidation of NADH as measured in FIG. 5A. The dotted curve shows the cleavage of a dithiopyridine (DTP) substrate as a measure of protein disulfide-thiol interchange.

FIG. 6 shows overexpression of tNOX in COS cells as determined after sodium dodecyl sulfate-polyacrylamide gel electrophoresis. The first two lanes (from left) are the results of Ponceau staining (lane 1, tNOX cDNA cloned into the pcDNA3.1 expression vector and transfected and expressed in COS cells; lane 2, vector without insert). The remaining lanes are the results of Western blotting with tNOX-specific antibody and detection (lane 3, tNOX cDNA; lane 4, vector without insert).

FIG. 7 graphically illustrates that the diameters of transfected COS cells were greater (approximately two times greater than those of untransfected COS cells).

FIG. 8 compares periodic changes in rates of cell enlargement (growth) of COS cells transfected with vector without insert (upper curve) and COS cells transfected with vector containing the tNOX cDNA insert (lower curve). The tNOX cDNA-transfected COS cells enlarge at about twice the rate of the control cells.

FIG. 9 shows that the COS cells transfected with the tNOX cDNA were more susceptible to capsaicin, which is a known anticancer agent and tNOX inhibitor.

FIG. 10 demonstrates that COS cells transfected with the tNOX cDNA were more susceptible to epigallocatechin gallate (EGCg), the principal anticancer constituent of green tea.

### DETAILED DESCRIPTION OF THE INVENTION

Abbreviations used herein for amino acids are standard in the art: X or Xaa represents an amino acid residue that has not yet been identified but may be any amino acid residue including but not limited to phosphorylated tyrosine, threonine or serine, as well as cysteine or a glycosylated amino acid residue. The abbreviations for amino acid residues as used herein are as follows: A, Ala, alanine; V, Val, valine; L, Leu, leucine; I, Ile, isoleucine; P, Pro, proline; F, Phe, phenylalanine; W, Trp, tryptophan; M, Met, methionine; G, Gly, glycine; S, Ser, serine; T, Thr, threonine; C, Cys, cysteine; Y, Tyr, tyrosine; N, Asn, asparagine; Q, Gln, glutamine; D, Asp, aspartic acid; E, Glu, glutamic acid; K, Lys, lysine; R, Arg, arginine; and H, His, histidine.

Additional abbreviations used herein include Mes, 2-(N-morpholino)ethanesulfonic acid; DMSO, dimethylsulfoxide; tNOX, cancer-associated and drug- (capsaicin-) responsive cell surface NADH oxidase; ttNOX, truncated tNOX; CNOX, constitutive and drug-unresponsive cell surface NADH oxidase; SDS-PAGE, sodium dodecylsulfate-polyacrylamide gel electrophoresis; capsaicin, 8-methyl-N-vanillyl-6-noneamide; LY181984, N-(4-methylphenylsulfonyl)-N′-(4-chlorophenyl)urea; LY181985, N-(4-methylphenylsulfonyl)-N′-(4-phenyl)urea; EGCg, (−)-epigallocatechin gallate.

As used herein, neoplasia describes a disease state of a human or an animal in which there are cells and/or tissues which proliferate abnormally. Neoplastic conditions include, but are not limited to, cancers, sarcomas, tumors, leukemias, lymphomas, and the like. The cell surface NADH oxidase/protein disulfide-thiol interchange protein of the present invention characterizes neoplastic cells and tissue as well as virus-infected cells (for example, human immunodeficiency virus, feline immunodeficiency virus, etc).

The cell surface marker which is characteristic of diseased cells is described in U.S. Pat. No. 5,605,810, issued Feb. 25, 1997, which is incorporated by reference herein, and in several scientific publications of which D. James Morré is sole author or a coauthor. This NADH oxidase/thiol interchange protein is found in the plasma membrane of neoplastic cells and cells infected with viruses, especially retroviruses and protozoan parasites. This protein is termed tNOX herein (tumor NADH oxidase). The cell surface tNOX protein is shed into serum and urine in cancer patients, but purification is relatively difficult. Therefore, it was a goal of the present work to obtain a cDNA clone encoding tNOX for use in recombinant production of the tNOX protein and for use of the tNOX coding sequences or portions thereof in probes and primers for the detection of tNOX transcripts or genomic sequences.

Immunological screening of a HeLa cell cDNA library using a tNOX-specific monoclonal antibody generated five clones. Restriction digestions were consistent with the derivation of all five clones from a single primary phage clone. That all five were inserts of different lengths of the same DNA was confirmed by automated nucleotide sequencing. The largest clone contained a 3.8-kb insert and an open reading frame of 1,830-bp (from nucleotide 23-1852 in SEQ ID NO: 1).

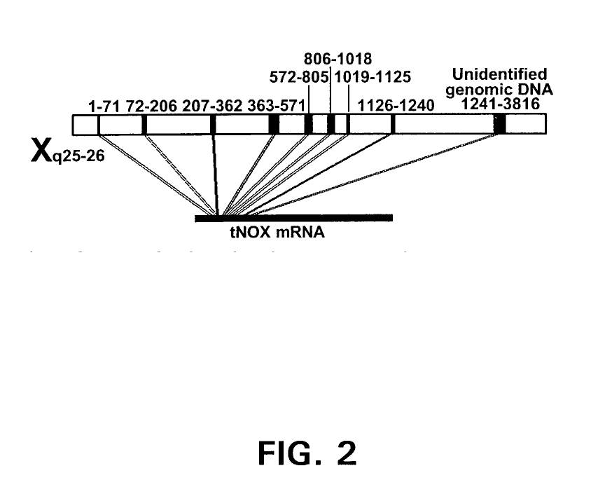

The full length cDNA yielded an open reading frame for a deduced amino acid sequence for a protein of 610 amino acids, with a predicted molecular weight of 70.1 kDa (Table 1). It contains a typical Kozak sequence (AXXATG) which facilitates translational expression (Kozak, 1987) at nucleotide 20. The initiator methionine at nucleotide 23 is followed at F5 by a sequence of 12 hydrophobic residues that serves as a signal sequence for membrane association. The termination codon at nucleotide 1853 is followed by a typical polyadenylation signal (AATAAA) at nucleotide 3625. Based on available genomic information (Bird, 1999), tNOX cDNA is comprised of multiple exons (at least nine) in just the N-terminal portion of the full-length precursor (FIG. 2).

The C-terminal portion of the derived amino acid sequence corresponded to the mature, processed MW of 34 kDa (ca 33.5 kDa from serum) as documented in previous studies (Morré et al., 1995a, 1996a; Chueh et al., 1997; del Castillo Olivares et al., 1998). Several potential functional motifs required of tNOX were contained in this portion of the protein as follows: The sequence E394-E-M-T-E forms a putative quinone binding site with 4 of 5 amino acids conserved (Table 2). The C505-X—X—X—X—C510 motif represents a potential active site for the protein disulfide-thiol interchange activity based on site-directed mutagenesis (Table 3). Also representing a potential active site for protein disulfide-thiol interchange activity from site-directed mutagenesis and from inhibition of activity by antisera to a C—X—X—X—X—X—C-containing peptide (LAILPACATPATCNPD) is C569-X—X—X—X—X—C575 (amino acids 569-575 of SEQ ID NO: 2).

The sequence T590-G-V-G-A-S-L (amino acids 590-595 of SEQ ID NO: 2) together with E605 forms a putative binding site for the adenine portion of NADH with 5 of 7 amino acids conserved with known mitochondrial adenine-binding proteins (Leblanc et al, 1995). The H546-V—H motif conserved in periplastic copper oxidases together with His467 form a potential copper binding ligand. In addition, the H546-V—H-E-F-G motif (amino acids 546-551 of SEQ ID NO: 2) is conserved in both human and chicken superoxide dismutase where it provides a putative copper-binding site (Shininá et al., 1996). Copper analyses by atomic absorption spectroscopy revealed at least 1 mole copper per 34 kDa processed tNOX subunit of the protein purified from sera of cancer patients.

Potential N-glycosylation sites (NXS/T) were at positions 138, 358, 418 and 525. Potential O-glycosylation sites include a threonine at amino acid 38, a threonine at amino acid 71, a serine at amino acid 35 and a serine at amino acid 240.

tNOX is a membrane-associated protein. Three putative signal sequences and cleavage sites near the N-terminus were identified as involved in membrane targeting. The second signal sequence near M220 would yield a 45.6 kDa protein containing all of the above identified functional motifs. The third potential signal sequence near M314 would result in a 34 kDa protein. In vitro translation of the cDNA of truncated tNOX starting at M220 using a rabbit reticulocyte lysate in the presence and absence of dog pancreatic microsomal membranes showed no indication of membrane insertion or apparent change in molecular weight of the in vitro translated product indicative of membrane-dependent processing. The truncated tNOX is encoded in SEQ ID NO: 1, nucleotides 680-1852.

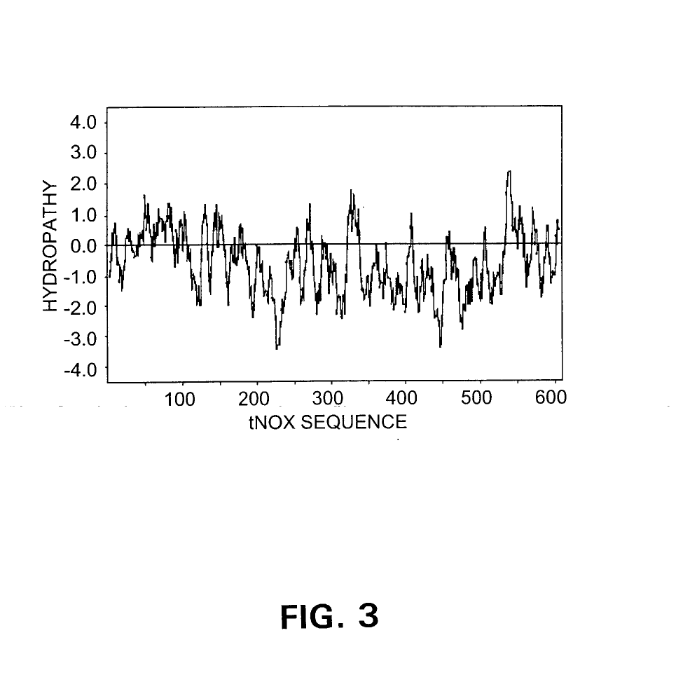

tNOX is a non-lipid-linked, extrinsic protein of the external plasma membrane surface (Morré, 1995). It is released from membranes by incubation at pH 5 (del Castillo et al., 1998). The hydropathy plot of the derived amino acid sequence of tNOX does not predict membrane-spanning domains (FIG. 3).

Because the deduced amino acid sequence of the tNOX protein (Table 1) showed homology over part of its length with the deduced amino acid sequence of a cDNA previously designated as APK1 antigen (from K357 to T610 of tNOX, amino acids 357-610 of SEQ ID NO: 2) (Chang and Pastan, 1994), the question arose, are tNOX and the K1 antigen the same proteins? The APK1 antigen cDNA sequence was obtained originally by expression cloning using a K1 antibody produced from the ovarian carcinoma cell line (OVCAR-3) as immunogen. A portion of the cDNA of tNOX appears to be the same as that isolated by Chang and Pastan except that their sequence contained one extra T at nucleotide 929 and one less G at nucleotide 1092 (at the nucleotide 83 and 247 of their sequence). These differences generated an incorrect reading frame. The two errors were confirmed by Sugano et al. (2000). The monoclonal antibody used for cDNA screening did not react with the K1 antigen expressed by OVCAR-3 cells nor do any of the properties of tNOX parallel those of the K1 antigen. The non-identity of tNOX and K1 antigen is consistent with a subsequent identification of the CAK1 protein as the protein reactive with the K1 antibody (Chang et al., 1992; Chang and Pastan, 1994).

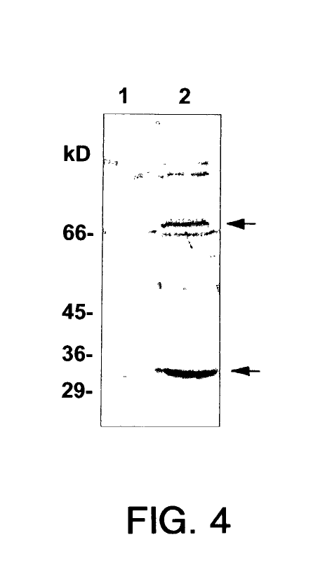

Neither the cell surface- or serum-derived nor the expressed tNOX share significant characteristics with the K-1 antigen. A high titer polyclonal antibody to the recombinant tNOX reacted with unprocessed (70 kDa) and processed (34 kDa) forms of tNOX expressed by OVCAR cells but failed to show any reactivity in portions of the gel corresponding to molecular weights of 30 kDa (APK1 antigen) or 40 kDa (CAK1) either in detergent solubilized (FIG. 4) or unsolubilized fractions. The CAK1 protein is expressed primarily in cell lines of mesothelial origin (Chang et al., 1992) and is anchored in the membrane by a glycosidic phosphatidyl-inositol (GPI) anchor. By contrast, tNOX lacks a GPI anchor.

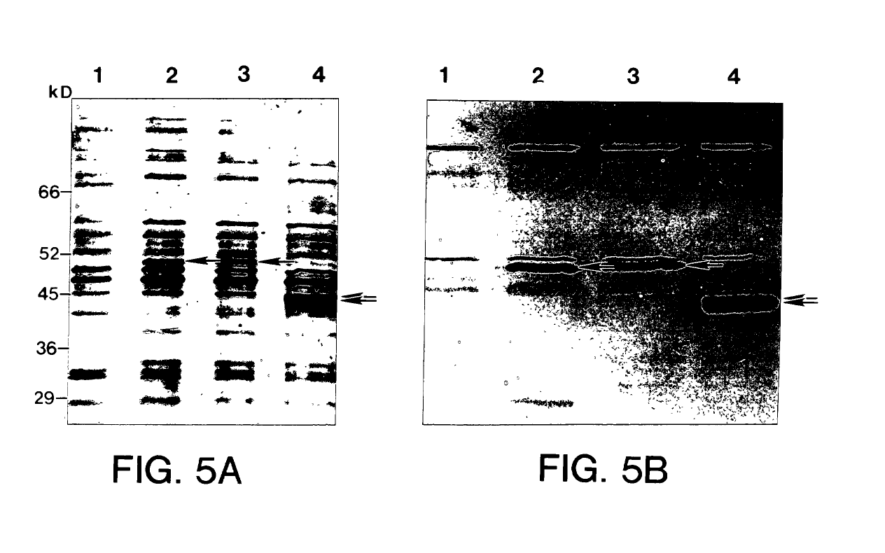

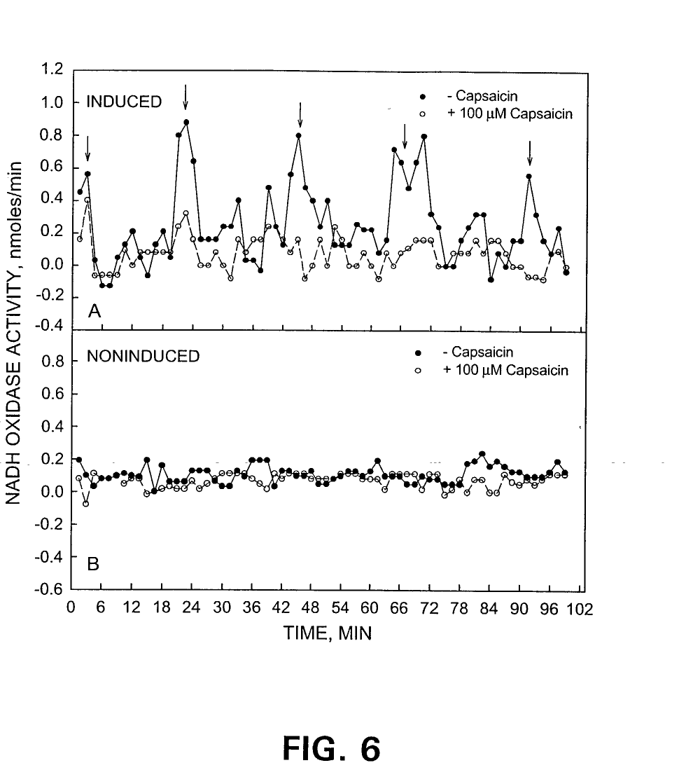

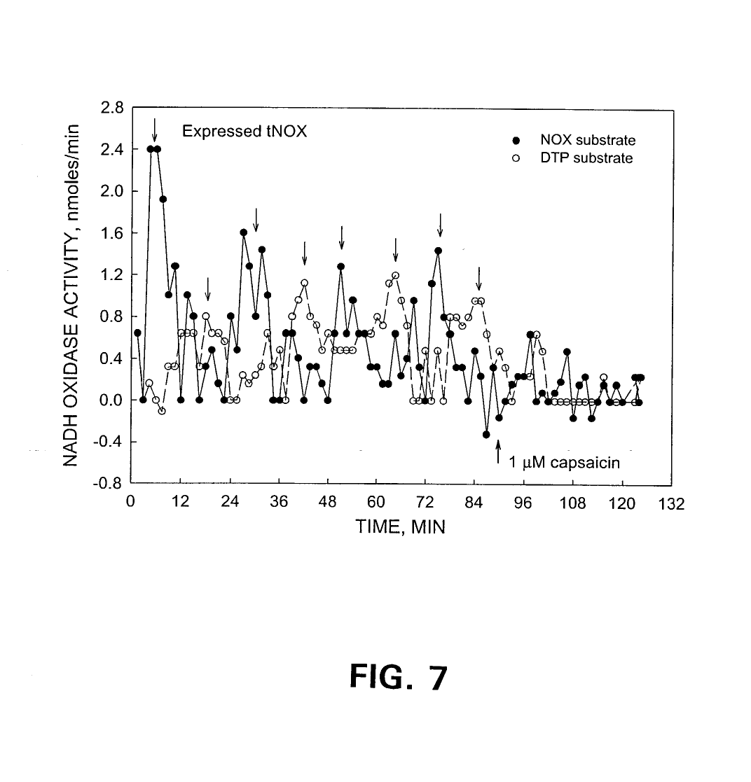

The expression of the tNOX cDNA in E. coli resulted in several forms of tNOX including a truncated 46 kDa beginning at M220 (ttNOX), 46 kDa histidine-tagged ttNOX and 34 kDa truncated tNOX beginning at G327. The entire sequence of the subcloned cDNA expressed in E. coli was confirmed by resequencing. tNOX proteins were identified by reaction with the tNOX-specific monoclonal antibody (FIG. 5). The apparent molecular weight of the ttNOX of 48 kDa on SDS-PAGE was consistent with the calculated molecular weight from the deduced amino acid sequence of 46 kDa. The molecular weight of the truncated tNOX beginning at G327 was 42 kDa on SDS-PAGE. Direct amino acid sequencing has revealed that the expressed protein purified from bacterial extract matched the deduced amino acid sequence. The induced bacterial extract exhibited a NADH oxidase activity with a 23 min period (arrows in FIG. 6A). Both the induced bacterial extracts when measured in the presence of 1 or 100 μM capsaicin (open circles in FIG. 6A and FIG. 7) or the uninduced extracts (FIG. 6B) had no periodic activity. The addition of 1 μM antitumor sulfonylurea LY181984 also completely inhibited the activity.

Illustrated in FIG. 7 is a second unique feature of the cell surface tNOX activity whereby the maximum rates of the two activities associated with the cloned and expressed protein, the hydroquinone (NADH) oxidase activity and the protein disulfide-thiol interchange (dithiodipyridine cleavage), alternate. As the rate of oxidation of NADH declines, the rate of DTDP cleavage increases, so that DTDP cleavage is at a maximum when NADH oxidation is at a minimum. Both had approximately the same period length of 23 min.

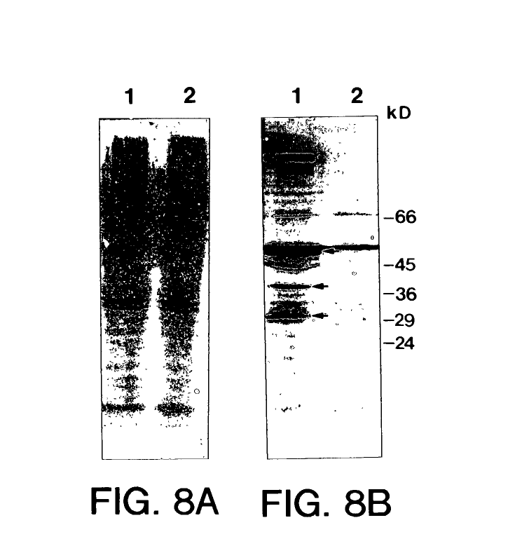

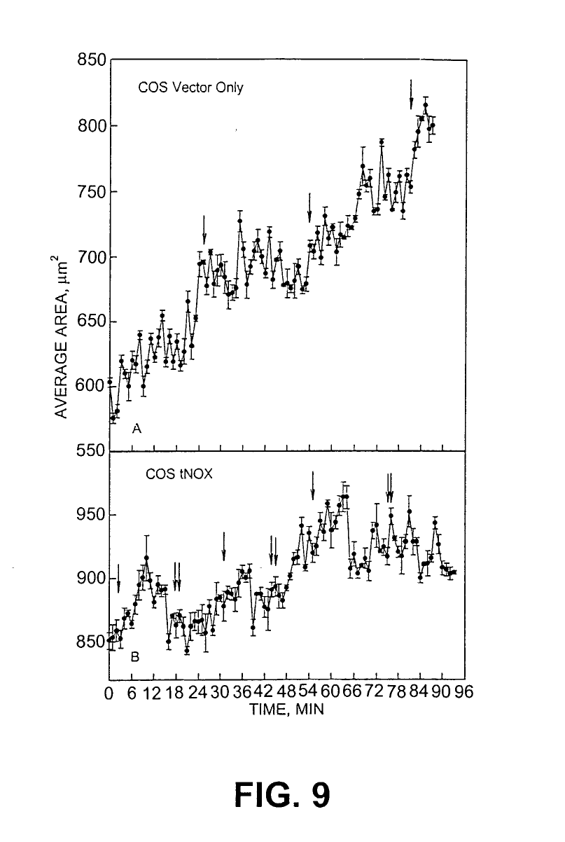

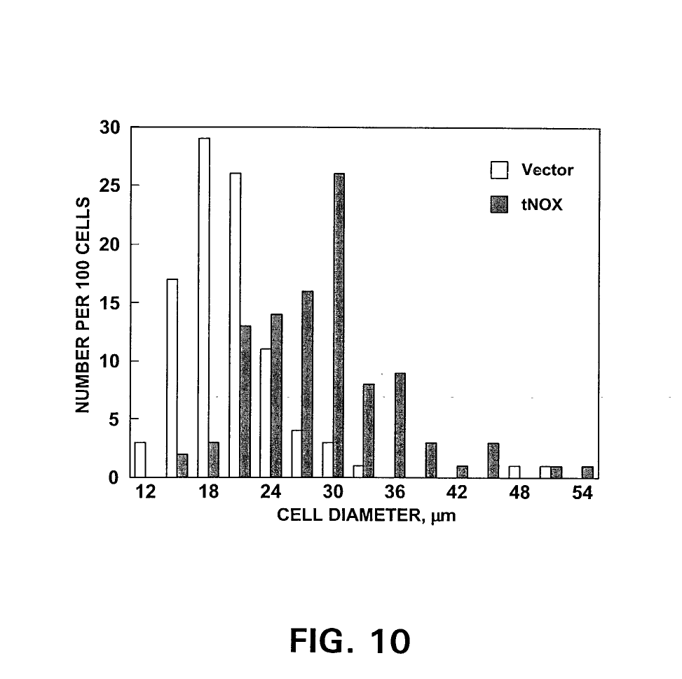

Peptide antisera against the tNOX C-terminus recognized expressed a truncated protein species (produced in recombinant COS-1 cells) with a molecular weight 48 kDa on SDS-PAGE (FIG. 8). Also present were two peptides of lower Mᵣ. Growth rates determined by image enhanced light microscopy of the ttNOX-transfected cells were about 2-fold greater than with vector alone (FIG. 9). The increased growth rate also was reflected in increased cell size. At confluency, the mean cell diameter of tNOX-transfected COS cells was about 30 μm whereas the average cell diameter of COS cells transfected with vector alone was about 20 μm (FIG. 10). The larger cell diameter resulted in a 4- to 5-fold increase in cell volume. An increased cell surface of the transfected cells was confirmed by electron microscopy. In keeping with the characteristic drug responsiveness of the oxidative activity that defines tNOX and the close relationship of tNOX activity to the enlargement phase of cell growth (Pogue et al., 2000), growth of tNOX cDNA-transfected COS cells exhibited a 10- to 100-fold greater susceptibility to tNOX inhibitors compared to cells transfected with vector alone (Table 3). tNOX inhibitors included capsaicin, (−)-epigallocatechin gallate (EGCg), adriamycin, and the active antitumor sulfonylurea, LY181984 (N-(4-methylphenylsulfonyl)-N′-(4-chlorophenyl)urea) (Table 4). With all four inhibitors, the EC50 of growth inhibition was shifted by 1 to 2 orders of magnitude to lower drug concentrations as a result of tNOX cDNA-transfection. The inactive antitumor sulfonylurea, LY181985 (N-(4-methylphenylsulfonyl-N′-(4-phenyl)urea) which differs from LY181984 by a single chlorine did not inhibit with either cells transfected with tNOX cDNA or with control cells transfected with vector alone. Similarly, the growth response to the non-tNOX inhibitor methotrexate, an antifolate, was unaffected by tNOX cDNA transfection.

The conclusion that the recombinant tNOX protein and the 34 kD NOX protein isolated from sera represent the same protein derives, in part, from the collective properties that define the two proteins. These include two different enzymatic activities, hydroquinone (NADH) oxidation and protein disulfide-thiol interchange (FIGS. 6 and 7), together with an alternation of these two activities to generate a period length of 22 min (FIGS. 6 and 7, Table 3). Additionally, the activities of both proteins respond to the same series of quinone site inhibitors and antitumor drugs in situ as well as in solution (Tables 3 and 4). It is the latter property that defines tNOX and distinguishes tNOX from other NOX proteins lacking drug responsiveness.

As previously demonstrated (Chueh et al., 1997; del Castillo et al., 1998), the correctly folded and active NOX proteins are blocked to direct sequencing and to N-terminal sequencing and/or enzymatic or chemical cleavage. However, a direct sequence link between the monoclonal antibody antigen employed in the cloning and amino acid sequence deduced for the 34 kD processed NOX form from the cell surface has come from protein purification studies. An incompletely processed 38.5 kD protein that cross-reacted with the monoclonal antibody and was converted to the 34 kD form upon digestion with proteinase K has been isolated from the HeLa cell surface. The 38.5 kD protein yielded a partial N-terminal sequence which was consistent with that of the deduced amino acid sequence of tNOX as presented in SEQ ID NO: 2.

A further characteristic of NOX proteins is that the two activities, NADH oxidation and protein disulfide-thiol interchange, alternate every 12 min to generate a regular pattern of oscillations with a temperature compensated and entrainable period length of ca 24 min (FIG. 7). Compared to CNOX with a precise 22 min period length (Pogue et al., 2000), ttNOX had a shorter period of 23 min. Mutant ttNOX proteins with different cysteine to alanine replacements were expressed in E. coli. Of these, C505A and C569A no longer exhibited NADH oxidase activity. The four other cysteine mutants retained NADH oxidase activity but the period lengths were changed (Table 3). For C575A and C602A, the period length for both NADH oxidation and protein disulfide-thiol interchange was increased to 36 min. For C510A and C558A, the period length to 39 min.

Our work identified an unusual NADH oxidase activity of the cell surface and plasma membrane of plant and animal cells. While the physiological function of the oxidative portion of the NOX cycle is that of a hydroquinone oxidase (Kishi et al., 1999), the oxidation of external NADH provides a convenient measure of the enzymatic activity. Interest in these proteins derives not only from their plasma membrane location but also from their potential roles as time-keeping proteins (Wang et al., 1998) and a relationship between the oscillatory enzymatic activity and the enlargement phase of cell growth (Morré, 1998; Pogue et al., 2000). The NOX proteins are unique in that they exhibit two different activities, hydroquinone oxidation and protein disulfide-thiol interchange. The two activities alternate (Morré, 1998; Sun et al., 2000) to generate the ca 24 min period.

While several NOX forms may exist, this first NOX form to be cloned and identified is the cancer-specific form designated tNOX. tNOX differs from the constitutive CNOX form present in both cancer and non-cancer tissues in its sensitivity to several anticancer drugs and to thiol reagents. The response of tNOX activity to the quinone site inhibitor capsaicin was used to guide purification of the processed tNOX protein from sera of cancer patients, as the basis for the monoclonal antibody selection and eventually to confirm the identity of the cloned cDNA based on complete capsaicin-inhibition of the activity of the bacterially expressed protein (FIG. 6).

The monoclonal antibody to the capsaicin-inhibited NADH oxidase from sera of cancer patients unequivocally identified a single cDNA sequence encoding the antigen. The sequence was one previously attributed to a cytosolic protein, the APK1 antigen (Chang and Pastan, 1994). The APK1 antigen was considered to be the antigen recognized by a monoclonal antibody designated K1 that was produced by hybridoma cells from mice immunized with ovarian carcinoma (OVCAR-3) cells. The longest cDNA of the study of Chang and Pastan (1994) contained 2,444-bp with a 789-bp open reading frame that encoded a protein of 30.5 kDa. The cDNA isolated by Chang and Pastan, despite missing and extra bases that generated a different reading frame from ours, was most likely identical to tNOX cDNA.

The protein reactive with the K1 antibody was originally identified as CAK1 (Chang et al., 1992). CAK1 is a membrane-bound protein with a molecular weight of 40 kDa, whereas the expressed APK1 gene product generated a soluble cytosolic protein (Chang and Pastan, 1994). CAK1 is expressed in ovarian cancers and mesotheliomas as well as in normal mesothelial cells. It appears to be a differentiation antigen that is expressed on cancers derived from mesothelium, such as epithelioid type mesotheliomas and ovarian cancers. It is a protein very distinct from tNOX. Using the monoclonal antibody K1, they eventually isolated a 2,138-bp cDNA that encoded CAK1 (Chang and Pastan, 1996). The cDNA had an 1,884-bp open reading frame encoding a 69 kDa protein. The 69 kDa precursor was processed to the 40 kDa form and the protein was named mesothelin because it was characteristic of mesothelial cells. When the cDNA was transfected into COS and NIH3T3 cells, the antigen was found on the cell surface and could be released by treatment with phosphatidylinositol-specific phospholipase C. tNOX is not anchored at the plasma membrane by a GPI linkage nor is it released by treatment with a phosphatidylinositol-specific phospholipase C. Mesothelin (CAK1), while associated with the cell membrane via a glycosyl-phosphatidylinositol tail, is not shed into the sera of cancer patients nor does it appear in conditioned medium supporting the growth of cultured cells (Chang and Pastan, 1994). As described earlier, tNOX has been isolated both from culture media by the growth of HeLa cells (Wilkinson et al, 1996) and from sera of cancer patients (Chueh et al., 1997). Furthermore, no protein sequence homology was found between CAK1 and tNOX.

In previous experiments, we had successfully photoaffinity-labeled the tNOX protein by [³²P]NAD(H), indicating that it contained a NADH binding site. NOX activity also responds to adenine nucleotides (Morré, 1998b). The typical adenine nucleotide binding sequence motif (G-X-G-X—X-G) with downstream remote acidic amino acid residues D or E (Yano et al., 1997) is represented most closely by T589-G-V-G-A-S-L (amino acids 589-595 of SEQ ID NO: 2) and E605 near the C-terminus. This sequence resembles closely the sequence T-G-V-G-A-G-V-G (SEQ ID NO: 3) from mitochondrial ATP synthase protein 9 from Chondous crispus (Leblanc et al., 1995).

The NOX protein binds the antitumor sulfonylurea LY 181984 (Morré et al., 1995c) and activity is inhibited or stimulated depending on the redox environment of the protein (Morré et al., 1998b). Reduced coenzyme Q is readily oxidized by the protein (Kishi et al., 1999) and other substances such as capsaicin and adriamycin which inhibit the activity are considered to occupy quinone sites. Ubiquinone protects against the binding and activity inhibition by the sulfonylurea LY181984. Thus, the presence in the tNOX sequence of a motif indicative of quinone binding as well as binding of sulfonylureas and other molecules known to occupy quinone sites, was sought.

A site with a methionine-histidine pair has been suggested to be the quinone binding site of pyruvate oxidase (Grabau and Cronan, 1986) by analogy with several quinone binding proteins of the photosystem II complex of chloroplasts. All known urea and sulfonylurea herbicide inhibitors of photosystem II are directed to such sites (Duke, 1990). Based on these considerations, a preliminary consensus sequence for the amino acids surrounding the charged residues critical to sulfonylurea and quinone-binding site was determined to be A-M-H-G (SEQ ID NO: 4) or a closely related sequence (Table 2). Apparently arginine can substitute for the critical histidine. For example, the putative quinone-binding site of the D1 protein of a cyanobacterium (Synechococcus), contains the sequence E-T-M-R-E (SEQ ID NO: 5). A sequence similar to E-T-M-R-E sequence is present in the NADH ubiquinone dehydrogenase of chloroplasts. Serum albumins also bind sulfonylureas and their putative sulfonylurea binding sites are included in Table I as well. We found a sequence E-M-T-E (amino acids 395-398 of SEQ ID NO: 2) as a potential quinone site having neither H nor R in the 4th position but still with considerable similarity to other putative quinone and/or sulfonylurea-binding sites. The correctness of identification of this E-M-T-E sequence as the drug binding site is supported by findings from the mutation M396A, which retains NADH oxidase activity but lost inhibition by capsaicin (Table II).

The first demonstrations of the thiol interchange activity for the tNOX protein used as the principal criterion, the restoration of activity to reduced, denatured and oxidized (scrambled) yeast RNase through reduction, refolding under non-denaturing conditions and reoxidation to form a correct secondary structure stabilized by internal disulfide bonds (Morré et al., 1997c). The restoration of activity to scrambled yeast RNase was similar to that catalyzed by protein disulfide isomerases of the endoplasmic reticulum (Freedman, 1989) but was clearly due to an activity of a different protein. The activity was not altered by the presence of two different antisera to protein disulfide isomerases (Morré et al., 1997c). One was mouse monoclonal antibody (SPA-891) from StressGen Biotechnologies to protein disulfide isomerase from bovine liver (cross-reactive with PDI from human, monkey, rat, mouse and hamster cell lines). The other was a peptide antibody of our own derivation directed to the characteristic C—X—X—C motif common to most, if not all, members of the protein disulfide isomerase family of proteins (Sharrosh and Dixon, 1991) but absent from tNOX. A C—X—X—C motif is present as well in thioredoxin reductase and related proteins where it appears to catalyze the transfer of electrons in conjunction with bound flavin (Russel and Model, 1988; Ohnishi et al., 1995). In addition to lacking C—X—X—C, tNOX does not appear to contain bound flavin nor is its activity dependent upon addition of flavin (FAD or FMN). Thus, the protein disulfide-thiol interchange catalyzed by tNOX appears to be distinct from that of classic protein disulfide isomerases or thioredoxin reductases.

The redox active disulfide of thioredoxin reductase from the malaria parasite Plasmodium falciparum, however, was in a motif C88-X—X—X—X—C93 (Gilberger et al., 1997) similar to those found in tNOX. This motif together with a downstream His509 was shown to be a putative proton donor/acceptor. A second C535-X—X—X—X—C540 motif in the same protein was crucially involved in substrate coordination and/or electron transfer (Gilberger et al., 1998). As suggested by the site directed mutagenesis results for tNOX, four of the eight cysteines present in truncated tNOX may be functionally paired. Results from site-directed mutagenesis (Table II) show that C505A and C569A mutations exhibit loss of both NADH oxidase and protein disulfide thiol interchange activities (manuscript in preparation). Thus, these two motifs, C505-X—X—X—X—C510 and C569-X—X—X—X—X—C575, alone or together with downstream histidines, might serve as part of the tNOX active site. tNOX was tested early for thioredoxin reductase activity and none was found. Despite the fact that tNOX lacks the two C—X—X—X—X—C motifs characteristic of flavoproteins, the sequence C505-A-S—R-L-C510 (amino acids 505-510 of SEQ ID NO: 2) or the sequence C569-T-S-D-V-E-C575 (amino acids 569-575 of SEQ ID NO: 2) might represent potential protein disulfide-thiol interchange motifs.

The remaining four cysteine mutations analyzed thus far exhibit an altered period length for the oscillations in tNOX activity (Table II) where both NADH oxidation and protein disulfide thiol interchange appear to be affected in parallel. The period length was increased from 23 min to 36 min for C575A and C602A whereas for C510A and C558A, the period length was increased to 36 min. Of potential interest is the observation that the 6-amino acid motif M588-T-G-V-G-A (amino acids 588-593 of SEQ ID NO: 2) of tNOX is shared with the Drosophila melanogaster clock period protein (Kliman and Hey, 1993).

At least under certain conditions, the tNOX protein catalyzes the transfer of electrons and protons to molecular oxygen. Oxygen uptake by plasma membranes prepared from HeLa cells is inhibited by the antitumor sulfonylurea LY181984 with approximately the same dose response (see Morré et al., 1998a) as other aspects of tNOX activity (see also Morré et al., 1998a). Therefore, we assume that tNOX and NOX proteins in general bind oxygen. The minimum requirement for an oxygen site would appear to be a metal together with appropriate covalent interactions such as hydrogen bonding (MacBeth et al., 2000). There are no indications that they might form a cluster with a typical motif for a [4Fe-4S] cluster binding site (C—X—X—C—X—X—C) and a remote cysteine followed by a proline. tNOX does contain a conserved copper site, which could provide the basis for oxygen binding.

The expression of truncated tNOX in E. coli and COS cells has confirmed that the cloned cDNA indeed exhibits fully the characteristics of the tNOX protein. All forms of tNOX (including the truncated and processed forms) were recognized by the tNOX-specific monoclonal antibody used in expression cloning. In addition, the expressed protein exhibited both enzymatic activities associated with NOX proteins (FIGS. 6 and 7). Overexpression of the tNOX proteins in COS cells stably transfected with the tNOX cDNA imparted tNOX-specific characteristics to the COS cells. The tNOX cDNA-transfected cells exhibited a 1.5 to 2-fold increase in cell size compared to control cells (3- to 5-fold increase in cell volume) and one to two log orders increase in sensitivity to tNOX-inhibitory drugs including capsaicin, (−)-epigallocatechin gallate (EGCg), adriamycin and the antitumor sulfonylureas (Table III). EGCg is the principal catechin responsible for the effects of green tea and green tea extracts on cancer prevention and on growth of cancer cells in culture (Chang, 2000). As is characteristic of other NOX inhibitors, EGCg inhibits the activity of tNOX but is largely without effect on the constitutive CNOX (Morré et al., 2000).

Taken together, the findings discussed herein confirm the molecular cloning and expression of the tNOX protein. The availability of the cDNA and the expressed protein will greatly facilitate future studies of the potential contribution of tNOX to unregulated growth and loss of differentiated characteristics linked to cancer.

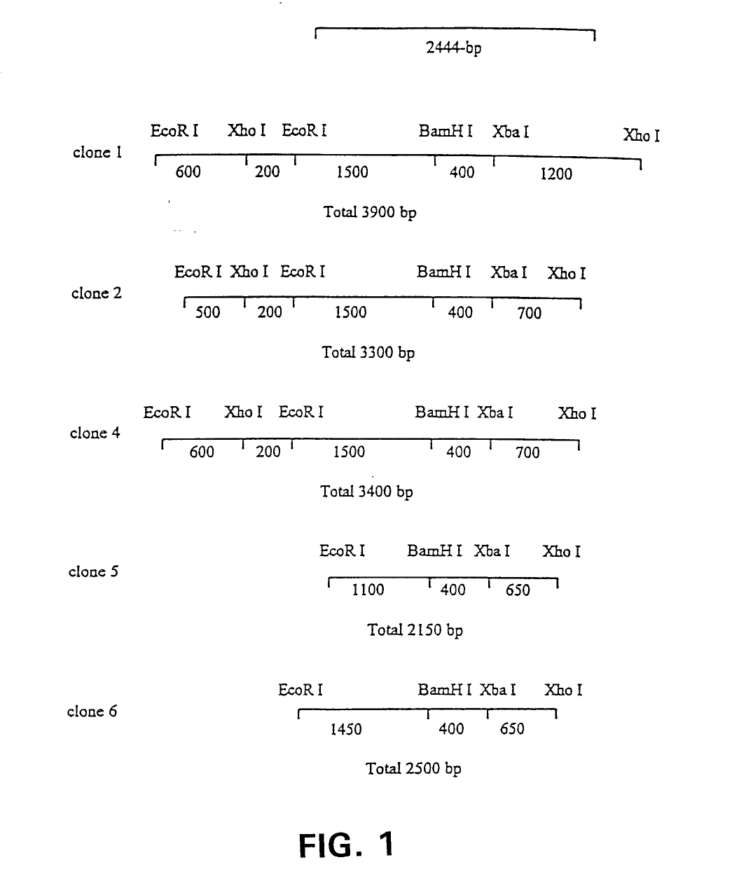

Primary screening of the commercially available HeLa cell cDNA library was performed by selecting from a total of sixteen 150-mm plates. Five positive clones (clone 1, 2, 4, 5 and 6; clone 3 was concluded to be false positive at secondary screening) were identified and further purified through at least three rounds of screening. Subsequently, in vivo excision was performed rather than subcloning because of its convenience and speed. Clone 1 contained the longest DNA insert with approximately 3,900-bp while clone 5 contained the shortest DNA insert with about 2,000-bp (FIG. 1). Several restriction endonucleases were utilized to determine the restriction sites (FIG. 1). The Uni-Zap XR library used in this study was constructed with EcoRI and XhoI double digestion. However, the digestion with EcoRI or XhoI alone demonstrated that there were both an internal EcoRI site and an XhoI site near the 5′ end of antisense strand in clone 1, clone 2 and clone 4. The lack of the internal EcoRI and XhoI sites in both clone 5 and clone 6 indicated that the DNA inserts in these two clones were further downstream with shorter 3′ ends. In addition, all of the five clones contained internal BamHI and XbaI sites. The double digestion of these two enzymes of each clone all produced a small (ca. 400 bp) segment of DNA. This phenomenon verified that those sites were identical in all five clones. The restriction mapping revealed that the five independent clones were identical except for the different lengths of DNA inserts. Since clone 1 contained the longest DNA insert, it was chosen for complete DNA sequencing. The rest of the four clones were sent for one round of automated sequencing. DNA sequences of all five clones were examined in the GenBank to seek identity or relatedness with other known genes. A computer-assisted search revealed that all five clones were similar to a DNA sequence designated as APK1 antigen [Chang and Pastan (1994) supra]. When all five of our clones were compared with the nucleotide sequence of APK1 antigen, two possible differences were observed in positions 83 and 246 of the APK1 antigen sequence. These two differences caused a shift in the open reading frame and in the deduced amino acid sequence.

The nucleotide sequence encoding human tNOX, recombinant human tNOX protein and recombinant cells which express recombinant human tNOX can be used in the production of recombinant tNOX for use in pancancer diagnostic protocols and as a target for (screening) new anticancer drugs.

It is understood by the skilled artisan that there can be limited numbers of amino acid substitutions in a plasma membrane NADH oxidase protein without significantly affecting function, and that nonexemplified plasma membrane NADH oxidase of neoplastic mammalian cells, virus- or parasite-infected mammalian cells or capsaicin-responsive plant plasma membrane NADH oxidase proteins can have some amino acid sequence divergence from the specifically exemplified amino acid sequence. Such naturally occurring variants can be identified, e.g., by hybridization to the exemplified coding sequence (or a portion thereof capable of specific hybridization to human tNOX sequences) under conditions appropriate to detect at least about 70% nucleotide sequence homology, preferably about 80%, more preferably about 90% or 95-100% sequence homology. Preferably the encoded tNOX has at least about 90% amino acid sequence identity to the exemplified tNOX amino acid sequence. In examining nonexemplified sequences, demonstration of the characteristic plasma membrane NADH oxidase and protein thiol interchange activities and the sensitivity of those activities to inhibitors such as capsaicin allows one of ordinary skill in the art to confirm that a functional protein is produced.

Also within the scope of the present invention are isolated nucleic acid molecules comprising nucleotide sequences encode tNOX proteins and which hybridize under stringent conditions to a nucleic acid molecule comprising the nucleic acid sequence of SEQ ID NO: 1 or a sequence corresponding to nucleotides 23 to 1852 thereof. DNA molecules with at least 85% nucleotide sequence identity to a specifically exemplified tNOX coding sequence sequence of the present invention are identified by hybridization under stringent conditions using a probe as set forth herein. Stringent conditions involve hybridization at a temperature between 65 and 68C. in aqueous solution (5×SSC, 5× Denhardt's solution, 1% sodium dodecyl sulfate) or at about 42C. in 50% formamide solution, with washes in 0.2×SSC, 0.1% sodium dodecyl sulfate at room temperature, for example. The ability of a sequence related to the specifically exemplified tNOX sequence of the present invention are readily tested by one of ordinary skill in the art.

As used in the present context, percent homology or percent sequence identity of two nucleic acid molecules is determined using the algorithm of Karlin and Altschul (1990) Proc. Natl. Acad. Sci. USA 87, 2264-2268, modified as described in Karlin and Altschul (1993) Proc. Natl. Acad. Sci. USA 90, 5873-5877. Such an algorithm is incorporated into the NBLAST and XBLAST programs of Altschul et al. (1990) J. Mol. Biol. 215, 402-410. BLAST nucleotide searches are performed with the NBLAST program, scor=100, wordlength=12, to obtain nucleotide sequences homologous to the nucleotide sequences of the present invention. BLAST protein searches are performed with the XBLAST program, score=50, wordlength=3, to obtain amino acid sequences homologous to a reference polypeptide sequence. To obtain gapped alignments for comparison purposes, Gapped BLAST is utilized as described in Altschul et al. (1997) Nucl. Acids Res. 25, 3389-3402/When using BLAST and Gapped BLAST programs, the default parameters of the respective programs (XBLAST and NBLAST) are used. Gaps introduced to optimize alignments are treated as mismatches in calculating identity. See, e.g., National Center for Biotechnology Information website on the internet.

It is well known in the biological arts that certain amino acid substitutions can be made in protein sequences without affecting the function of the protein. Generally, conservative amino acids are tolerated without affecting protein function. Similar amino acids can be those that are similar in size and/or charge properties; for example, aspartate and glutamate and isoleucine and valine are both pairs of similar amino acids. Similarity between amino acid pairs has been assessed in the art in a number of ways. For example, Dayhoff et al. [(1978) In: Atlas of Protein Sequence and Structure, Volume 5, Supplement 3, Chapter 22, pp. 345-352], which is incorporated by reference herein, provides frequency tables for amino acid substitutions which can be employed as a measure of amino acid similarity. Dayhoff et al.'s frequency tables are based on comparisons of amino acid sequences for proteins having the same function from a variety of evolutionarily different sources. The art provides methods for determining tNOX activity, including its characteristic response to certain inhibitors (capsaicin, adriamycin, quassinoids, etc).

A polynucleotide or fragment thereof is substantially homologous (or substantially similar) to another polynucleotide if, when optimally aligned (with appropriate nucleotide insertions or deletions) with another polynucleotide, there is nucleotide sequence identity for approximately 60% of the nucleotide bases, usually approximately 70%, more usually about 80%, preferably about 90%, and more preferably about 95% to 100% of the nucleotide bases.

Alternatively, substantial homology (or similarity) exists when a polynucleotide or fragment thereof will hybridize to another polynucleotide under selective hybridization conditions. Selectivity of hybridization exists under hybridization conditions which allow one to distinguish the target polynucleotide of interest from other polynucleotides. Typically, selective hybridization will occur when there is approximately 55% similarity over a stretch of about 14 nucleotides, preferably approximately 65%, more preferably approximately 75%, and most preferably approximately 90%. See Kanehisa [(1984) Nucl. Acids Res. 12:203-213]. The length of homology comparison, as described, may be over longer stretches, and in certain embodiments will often be over a stretch of about 17 to 20 nucleotides, and preferably about 36 or more nucleotides. The hybridization of polynucleotides is affected by such conditions as salt concentration, temperature or organic solvents, in addition to the base composition, length of the complementary strands, and the number of nucleotide base mismatches between the hybridizing polynucleotides, as will be readily appreciated by those skilled in the art. However, the combination of parameters is much more important than the measure of any single parameter [Wetmur and Davidson (1968) J. Mol. Biol. 31:349-370].

An isolated or substantially pure polynucleotide is a polynucleotide which is substantially separated from other polynucleotide sequences which naturally accompany a native tNOX protein coding sequence. The term embraces a polynucleotide sequence which has been removed from its naturally occurring environment, and includes recombinant or cloned DNA isolates, chemically synthesized analogues and analogues biologically synthesized by heterologous systems.

A polynucleotide is said to encode a polypeptide if, in its native state or when manipulated by methods known to those skilled in the art, it can be transcribed and/or translated to produce the polypeptide of a fragment thereof. The antisense strand of such a polynucleotide is also said to encode the sequence. The assay methods described hereinbelow allow the confirmation that an active tNOX protein with intact response patterns to inhibitors of authentic tNOX is produced upon expression of the coding sequence disclosed herein in a recombinant host cell.

A nucleotide sequence is operably linked when it is placed into a functional relationship with another nucleotide sequence. For instance, a promoter is operably linked to a coding sequence if the promoter affects its transcription or expression. Generally, operably linked means that the sequences being linked are contiguous and, where necessary to join two protein coding regions, contiguous and in reading frame. However, it is well known that certain genetic elements, such as enhancers, may be operably linked even at a distance, i.e., even if not contiguous.

The term non-naturally occurring or recombinant nucleic acid molecule refers to a polynucleotide which is made by the combination of two otherwise separated segments of a sequence accomplished by the artificial manipulation of isolated segments of polynucleotides by genetic engineering techniques or by chemical synthesis. In so doing one may join together polynucleotide segments of desired functions to generate a desired combination of functions.

Polynucleotide probes include an isolated polynucleotide attached to a label or reporter molecule and may be used to identify and isolate other tNOX protein coding sequences. Probes comprising synthetic oligonucleotides or other polynucleotides may be derived from naturally occurring or recombinant single- or double-stranded nucleic acids or be chemically synthesized. They may be used in polymerase chain reactions as well as in hybridizations. Polynucleotide probes may be labeled by any of the methods known in the art, e.g., random hexamer labeling, nick translation, or the Klenow fill-in reaction. Oligonucleotides or polynucleotide primers useful in PCR are readily understood and accessible to the skilled artisan using the sequence information provided herein taken with what is well known to the art.

Large amounts of the polynucleotides may be produced by replication in a suitable host cell. Natural or synthetic DNA fragments coding for a tNOX protein incorporated into recombinant polynucleotide constructs, typically DNA constructs, capable of introduction into and replication in a prokaryotic or eukaryotic cell, desirably a yeast cell, and preferably a Saccharomyces cerevisiae cell are provided by the present invention. Usually the construct will be suitable for replication in a unicellular host, such as yeast or bacteria, but a multicellular eukaryotic host may also be appropriate, with or without integration within the genome of the host cells. Commonly used prokaryotic hosts include strains of Escherichia coli, although other prokaryotes, such as Bacillus subtilis or Pseudomonas may also be used. Yeasts suitable for the present invention include species of Saccharomyces and Pichia, e.g., Pichia pastoris. Mammalian (e.g., CHO or COS cells) or other eukaryotic host cells include filamentous fungi, plant, insect, amphibian and avian species. Such factors as ease of manipulation, ability to appropriately glycosylate expressed proteins, degree and control of protein expression, ease of purification of expressed proteins away from cellular contaminants, or other factors may determine the choice of the host cell. Vectors suitable for use in the foregoing host cells are well known to the art and are widely available in research laboratories as well as through commerce.

The polynucleotides may also be produced by chemical synthesis, e.g., by the phosphoramidite method described by Beaucage and Caruthers [(1981) Tetra. Letts. 22:1859-1862] or the triester method according to Matteuci et al. [(1981) J. Am. Chem. Soc. 103:3185], and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA tNOX protein with an appropriate primer sequence.

DNA constructs prepared for introduction into a prokaryotic or eukaryotic host cell typically comprise a replication system (i.e. vector) recognized by the host, including the intended DNA fragment encoding the desired polypeptide, and preferably also include transcription and translational initiation regulatory sequences operably linked to the tNOX protein-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides may also be included where appropriate from secreted polypeptides of the same or related species, which allow the protein to cross and/or lodge in cell membranes or be secreted from the cell.

An appropriate promoter and other necessary vector sequences will be selected so as to be functional in the host. Examples of workable combinations of cell lines and expression vectors are described in Sambrook et al. [(1989) vide infra; Ausubel et al. (Eds.) (1992) Current Protocols in Molecular Biology, Greene Publishing and Wiley Interscience, New York] and Metzger et al. [(1988) Nature 334:31-36]. Many useful vectors for expression in bacteria, yeast, mammalian, insect, plant or other cells are well known in the art and may be obtained from such vendors as Stratagene, New England Biolabs, Promega, and others. In addition, the construct may be joined to an amplifiable gene (e.g., DHFR) so that multiple copies of the gene may be made. For appropriate enhancer and other expression control sequences, see also Enhancers and Eukaryotic Gene Expression, Cold Spring Harbor Press, NY (1983). While such expression vectors may replicate autonomously, they may less preferably replicate by being inserted into the genome of the host cell.

Expression and cloning vectors desirably contain a selectable marker, that is, a gene encoding a protein necessary for the survival or growth of a host cell transformed with the vector. Although such a marker gene may be carried on another polynucleotide sequence co-introduced into the host cell, it is most often contained on the cloning vector. Only those host cells into which the marker gene has been introduced will survive and/or grow under selective conditions. Typical selection genes encode proteins that (a) confer resistance to antibiotics or other toxic substances, e.g., ampicillin, neomycin, methotrexate, etc.; (b) complement auxotrophic deficiencies; or (c) supply critical nutrients not available from complex media. The choice of the proper selectable marker will depend on the host cell; appropriate markers for different hosts are known in the art.

The coding sequence and the deduced amino acid sequence for the tNOX are provided in Table 1. See also SEQ ID NO: 1 and SEQ ID NO: 2.

A combination of restriction endonuclease cutting and site-directed mutagenesis via PCR using an oligonucleotide containing a desired restriction site for cloning (one not present in coding sequence), a ribosome binding site, a translation initiation codon (ATG) and the codons for the first amino acids of tNOX can be employed to engineer tNOX for recombinant expression. Site-directed mutagenesis strategy is described, for example, in Boone et al. [(1990) Proc. Natl. Acad. Sci. USA 87:2800-2804], with modifications for use with PCR as readily understood by the skilled artisan.

The skilled artisan understands that it may be advantageous to modify the exemplified tNOX coding sequence for improved expression in a particular recombinant host cell. Such modifications, which can be carried out without the expense of undue experimentation using the present disclosure taken together with knowledge and techniques readily accessible in the art, can include adapting codon usage so that the modified tNOX protein coding sequence has codon usage substantially like that known for the target host cell. Such modifications can be effected by chemical synthesis of a coding sequence synonymous with the exemplified coding sequence or by oligonucleotide site-directed mutagenesis of selected portions of the coding sequence.

Compositions and immunogenic preparations, including vaccine compositions, comprising substantially purified recombinant tNOX virus or an immunogenic peptide having an amino acid sequence derived therefrom and a suitable carrier therefor are provided by the present invention. Alternatively, hydrophilic regions of the tNOX can be identified by the skilled artisan, and peptide antigens can be synthesized and conjugated to a suitable carrier protein (e.g., bovine serum albumin or keyhole limpet hemocyanin) if needed for use in vaccines or in raising polyclonal or monoclonal antibodies specific for the exemplified tNOX. Immunogenic compositions are those which result in specific antibody production when injected into a human or an animal. The vaccine preparations comprise an immunogenic amount of the exemplified tNOX or an immunogenic fragment(s) thereof. Such vaccines may comprise tNOX, alone or in combination with another protein or other immunogen. By “immunogenic amount” is meant an amount capable of eliciting the production of antibodies directed against the exemplified tNOX in an individual or animal to which the vaccine has been administered.

Immunogenic carriers can be used to enhance the immunogenicity of the tNOX or peptides derived in sequence therefrom. Such carriers include but are not limited to proteins and polysaccharides, liposomes, and bacterial cells and membranes. Protein carriers may be joined to the tNOX protein or peptides derived therefrom to form fusion proteins by recombinant or synthetic means or by chemical coupling. Useful carriers and means of coupling such carriers to polypeptide antigens are known in the art.

Preferred fusion proteins which are effective for stimulating an immune response, especially when administered orally (e.g., in food or water) include those fusion proteins with a cholera toxin fragment, or so-called LTB fusion. These methods are described in Dougan et al. [(1990) Biochem. Soc. Trans. 18:746-748] and Elson et al. [(1984) J. Immunol. 132:2736-2741].

The immunogenic compositions and/or vaccines may be formulated by any of the means known in the art. They are typically prepared as injectables, either as liquid solutions or suspensions. Solid forms suitable for solution in, or suspension in, liquid prior to injection may also be prepared. The preparation may also, for example, be emulsified, or the protein(s)/peptide(s) encapsulated in liposomes.

The active immunogenic ingredients are often mixed with excipients or carriers which are pharmaceutically acceptable and compatible with the active ingredient. Suitable excipients include but are not limited to water, saline, dextrose, glycerol, ethanol, or the like and combinations thereof. The concentration of the immunogenic polypeptide in injectable formulations is usually in the range of 0.2 to 5 mg/ml.

In addition, if desired, the vaccines may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-( 1′-2′-dipalmitoyl-sn-glycero-3-hydroxyphosphoryloxy)-ethylamine (CGP 19835A, referred to as MTP-PE); and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogen resulting from administration of the immunogen in vaccines which are also comprised of the various adjuvants. Such additional formulations and modes of administration as are known in the art may also be used.

tNOX as exemplified herein and/or epitopic fragments or peptides of sequences derived therefrom or from other tNOX proteins having primary structure similar (more than 90% identity) to the exemplified tNOX protein may be formulated into vaccines as neutral or salt forms. Pharmaceutically acceptable salts include but are not limited to the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine.

Multiantigenic peptides having amino acid sequences derived from the exemplified tNOX for use in immunogenic compositions are synthesized as described in Briand et al. [(1992) J. Immunol. Methods 156:255-265].

The immunogenic compositions or vaccines are administered in a manner compatible with the dosage formulation, and in such amount as will be prophylactically and/or therapeutically effective. The quantity to be administered, which is generally in the range of about 100 to 1,000 μg of protein per dose, more generally in the range of about 5 to 500 μg of protein per dose, depends on the subject to be treated, the capacity of the individual's immune system to synthesize antibodies, and the degree of protection desired. Precise amounts of the active ingredient required to be administered may depend on the judgment of the veterinarian, physician or doctor of dental medicine and may be peculiar to each individual, but such a determination is within the skill of such a practitioner. Especially for poultry, immunogenic compositions can be administered orally via food or water preparations comprising an effective amount of the protein(s) and/or peptide(s), and these immunogenic compositions may be formulated in liposomes as known to the art.

The vaccine or other immunogenic composition may be given in a single dose or multiple dose schedule. A multiple dose schedule is one in which a primary course of vaccination may include 1 to 10 or more separate doses, followed by other doses administered at subsequent time intervals as required to maintain and or reinforce the immune response, e.g., at 1 to 4 months for a second dose, and if needed, a subsequent dose(s) after several months.

Antibodies specific for the plasma membrane tNOX and the shed forms in the urine and serum of cancer patients and animals with neoplastic disorders are useful, for example, as probes for screening DNA expression libraries or for detecting or diagnosing a neoplastic disorder in a sample from a human or animal. Desirably the antibodies (or second antibodies which are specific for the antibody which recognizes tNOX) are labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. United States Patents describing the use of such labels include, but are not limited to, U.S. Pat. Nos. 3,817,837; 3,850,752; 3,939,350; 3,996,345; 4,277,437; 4,275,149; and 4,366,241. Antibodies useful in diagnostic and screening assays can be prepared using a peptide antigen whose sequence is derived from all or a part of SEQ ID NO: 2, for example, SEQ ID NO: 16, the full length protein or a protein corresponding to amino acids 220-610.

All references cited herein are hereby incorporated by reference in their entirety to the extent that they are not inconsistent with the present disclosure.

Except as noted hereafter, standard techniques for peptide synthesis, cloning, DNA isolation, amplification and purification, for enzymatic reactions involving DNA ligase, DNA tNOX protein, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold spring Harbor Laboratory, Cold Spring Harbor, N.Y., Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein.

The foregoing discussion and the following examples illustrate but are not intended to limit the invention. The skilled artisan will understand that alternative methods may be used to implement the invention.

### EXAMPLES

### Example 1

Materials and Bacterial Cultures.

The antigen of the monoclonal antibody was isolated as previously described [Chueh et al. (1997)]. Peroxidase-conjugated goat anti-mouse IgG (Jackson ImmunoResearch Laboratories, West Grove, Pa.) was used to form an antigen-antibody-antibody-AP color complex. E. coli strains XL1-blue and SOLR, a Uni-Zap XR HeLa cell cDNA library, helper phage and expression vector pET11 were purchased from Stratagene (La Jolla, Calif.). Luria-Bertani broth (LB broth) media and agar were supplied by DIFCO (Detroit, Mich.). DNA markers, restriction endonucleases and the plasmid DNA purification kit were purchased from Promega (Madison, Wis.). The mammalian expression system including expression vector pcDNA3. 1 was purchased from Invitrogen (Carlsbad, Calif.). Unless indicated otherwise, all chemicals were purchased from Sigma Chemical Co. (St. Louis, Mo.).

The recA⁻ E. coli host strain, XL1-blue, was first streaked on a 100 mm LB-tetracycline (12.5 μg/ml) agar plate, followed by overnight incubation at 37° C. One isolated colony was picked up by a sterile wire loop and then inoculated in LB-media at 37° C. The plate was wrapped in Parafilm and placed in a 4° C. refrigerator until the next streaking.

Fifty ml of LB broth was supplemented with 0.2% (v/v) maltose and 10 mM MgSO₄ in a sterile flask. The cells were grown overnight with gentle shaking at 37° C. At day 2, liquid culture was centrifuged in a sterile conical tube for 15 minutes at 4,000 rpm, followed by removal of the media from the cell pellet. The pellet was resuspended gently in 15 ml of 10 mM MgSO₄ solution. Subsequently, cells were diluted to an OD₆₀₀ of 0.5 with 10 mM MgSO₄ for later use. For every experiment, a new streak plate was used.

### Example 2

Generation of Monoclonal Antibody

The antigen utilized for the generation of the monoclonal antibody was isolated and characterized from pooled sera of cancer patients (Chueh et al., 1997). The fraction containing a ca 34 kDa protein with capsaicin-inhibited tNOX activity was concentrated with a Centricon (Amicon, Mass.) followed by washing with PBS three times to remove excess salts. The monoclonal antibody and hybridomas were generated in the Monoclonal Antibody Facility of the Purdue Cancer Center following standard protocols (Schook, 1987). Two BALB/c mice were immunized with tNOX protein mixed with complete Freund's adjuvant and boosted three times at 3-week intervals. Hybridomas were screened both by enzymatic activity assay and Western blot analysis. Antisera-generating clones with the following characteristics were selected: ability to block completely drug responsive NOX activity of cancer cells and sera of cancer patients, to immunoprecipitate the protein with capsaicin-inhibited NADH oxidase activity from the surface of cancer cells and of sera pooled from cancer patients, having no effect on the NADH oxidase activity of sera from healthy volunteers and reactive with a 34 kDa cell surface protein of HeLa cells and sera of cancer patients.

### Example 3

Isolation of the cDNA Clones.

The HeLa Uni-Zap cDNA library was first screened as described [Sambrook et al. (1989) supra] at approximately 50,000 plaque-forming units per 150 mm plate using monoclonal ascites (1:100 dilution) and peroxidase-conjugated goat anti-mouse IgG (1:50,000 dilution). Five positive plaques were isolated from a total of about 8×10⁵ total plaques screened and the bacteriophages were purified to homogeneity by at least three rounds of screening and selection. In vivo excision of the positive phage clones with ExAssist helper phage (M13) was then performed according to the protocol from Stratagene to convert the Uni-Zap plasmids to pBluescript phagemids. The circularized phagemid DNAs were extracted by utilizing Wizard Plus miniprep DNA purification kits according to the manufacturer's recommendations (Promega, Madison, Wis.). Restriction enzyme mapping using ExoRI, XhoI, and BamHI showed that all five clones were identical in origin. The tNOX insert was sequenced using T3′ and T7 primers. The complete nucleotide sequence of cDNA clone 1 was obtained using the gene walking technique and 10 17 bp synthetic primers (DNA Sequencing Service, Tufts University, Boston, Mass.). Searches within the NCBI/GenBank database were with nucleotide sequence and deduced amino acid sequence information for the longest open reading frame uncovered.

### Example 4

DNA Agarose Electrophoresis.

A 1.2% agarose gel was prepared by adding 0.9 g of agarose into 75 ml of TBE buffer (10.8 g Tris, 5.5 g boric acid and 0.93 g Na₂EDTA.2H₂O brought to 1 liter with distilled deionized water) and heated until all agarose was completely dissolved. TBE buffer was filtered before use. Ethidium bromide was added to the gel solution at a final concentration of 0.5 μg/ml solution before the gel solution was cast. Immediately, the mixture was poured onto the cast and a comb was placed in the proper position. The gel was cast at least for 30 minutes before electrophoresis. The comb was removed and the gel was placed into the electrophoresis system and TBE buffer was added until the gel was covered by buffer. Markers and DNA samples were mixed with loading buffer and pipetted into separate wells. The electrophoresis was performed at 90 V for approximately 1.5 hours.

### Example 5

Sequencing Analysis and Restriction Mapping.

Several restriction endonucleases (EcoRI, XhoI, BamHI, XbaI, KpnI and SalI) were utilized to determine restriction sites. The digestion was performed according to the protocol provided by Promega (Madison, Wis.). Eleven μl of H₂O, 2 μl of 10× reaction buffer, 2 μl of 1 μg/μl of BSA, 4 μl of DNA and 1 μl of the respective restriction endonuclease were mixed by pipetting into an eppendorf tube and centrifuging for several seconds. The mixture was incubated at the optimum temperature for three to four hours dependent on the enzyme. Subsequently, agarose electrophoresis was performed after each digestion. The DNA sequence was first analyzed by automated sequencing using T3 and T7 primers. The complete nucleotide sequence was determined on both DNA strands. The gene walking was performed by using 10 17-bp synthetic primers. The nucleotide sequences of all five clones and the deduced amino-acid sequence of clone 1 were analyzed for homology using BLAST and Pedro program against GenBank.

### Example 6

Expression of tNOX and Histidine-Tagged tNOX Proteins in Bacteria

tNOX cDNA from clone 1 was expressed in E. coli either as a truncated form (ttNOX) (beginning at M220), as a fusion protein with six histidine residues (ttNOX-his) fused to the amino terminus of ttNOX, or as a processed tNOX (beginning at G327). First, the open reading frame of ttNOX DNA and nucleotides of 3′-untranslated region were amplified by PCR, digested with NdeI and BamHI followed by ligation into the protein expression vector pET-11b. All primers were synthesized by the Laboratory for Macromolecular Structure (Purdue University, IN). Primers for PCR amplification of ttNOX were 5′-GAGTGTAAACAGCATATGCTAGCCAGA-3′ (forward, SEQ ID NO: 6) and 5′TTTCTATGCTTGTCCAACACATAT-3′ (reverse, SEQ ID NO: 7). Primers for processed form of tNOX were 5′-GGAGATATACATATGGGAATTCTCATTCAA-3′ (forward, SEQ ID NO: 8) and 5′-TTTCTATGCTTGTCCAACACATAT-3′ (reverse, SEQ ID NO: 9). Primers for histidine-tagged ttNOX were 5′-GATATACATATGCATCATCATCATCATCATCTAGCCAGAGAGGAGCGCCAT-3′ (forward, SEQ ID NO: 10) and 5′-TTTCTATGCTTGTCCAACACATAT-3′ (reverse, SEQ ID NO: 11). The forward primer was designed to incorporate six histidine residues to the amino terminus of tNOX protein. The amplification performed was with an initiation step of 94C. for 90 sec, followed by 90 sec of denaturation at 94C., 90 sec of annealing at 55C., and 90 sec of extension at 72C. for 29 cycles.

E. coli [BL21 (DE3)] were transfected and grown in LB medium containing ampicillin (100 μg/ml) for 16 hr at 25C. and harvested. DNA sequences of the ligation products were confirmed by DNA sequencing. Expressions of all forms of tNOX were confirmed by SDS-PAGE with silver staining and immunoblotting. Immunoblot analysis was with anti-tNOX monoclonal antibody. Detection used alkaline phosphate conjugated anti-mouse antibody.

### Example 7

Expression of ttNOX in COS Cells

Transient transfection of COS cells were with pcDNA3.1 (Invitrogen) and a Calcium Phosphate Transfection Kit (Invitrogen, Carlsbad, Calif.) according to the manufacturer's protocol. ttNOX cDNA was first amplified by PCR using primers 5═-TGGGAGTGTAAACAGCGTATG-3′ (forward; SEQ ID NO: 12) and 5′-TTTCTATGCTTGTCCAACACATAT-3′ (reverse, SEQ ID NO: 13). The PCR product was then amplified using primers 5′-AAACTTAAGCTTTGGGAGTGT-3′ (forward, SEQ ID NO: 14) and 5′-TTTCTATGCTTGTCCAACACATAT-3′ (reverse, SEQ ID NO: 15) to construct a HindIII site at 5′ end of the nontemplate strand. The product was double digested using HindIII and BamHI enzymes. The digested products were separated on an agarose gel and extracted using a DNA Extraction Kit (Qiagen, Valencia, Calif.). The DNA was then ligated into a pcDNA3.1 vector that contains a cytomegalovirus enhancer-promoter for high levels of expression. For propagation of the plasmid DNA, the ligation product was used to transform XL-1 blue competent cells using heat pulse technique (Sambrook et al., 1989, supra). The positive clones were identified by PCR. The resulting plasmid was then used to transfect COS cells.

COS-1 cells (African monkey kidney cell line), were plated one day prior to transfection at 4×10⁵ cells per 100-mm dish. Thirty-six μl of 2 M CaCl₂ and 30 μg of pcDNA3.1 or pcDNA3.1-tNOX in 300 μl sterile H2O were slowly added dropwise into 300 μl of 2× Hepes Buffered Saline (HBS) at room temperature for 30 min. The transfection mixtures then were added dropwise to the media to the cells and incubated overnight at 37C. After overnight exposure to the DNA precipitate, the cells were washed twice with PBS and 3 ml of DMSO were added for 2.5 min. The DMSO then was removed and cells were fed with fresh media for 2-3 days. tNOX expression was evaluated on the basis of enzymatic activity and Western blot analysis. For selection of stable transfectants, antibiotic G418 sulfate was used (Invitrogen, Carlsbad, Calif.). After the COS cells were transfected with the tNOX cDNA expression plasmid, 0.5 mg/ml of G418 sulfate was added into the media twice a week and the cultures were maintained until colonies 2 to 3 mm in diameter were formed. A total of three colonies were selected and trypsinized individually and then transferred into wells of a 24-well plate and then into a 35 mm petri dish. Cells were harvested at 80% confluency. Transfections were confirmed by immunoblotting.

### Example 8

N-Terminal Amino Acid Sequencing of Expressed tNOX

For partial amino acid sequencing, recombinant tNOX protein from the recombinant E. coli extract were precipitated with 20% ammonium sulfate, electrophoresed on 12% SDS-PAGE and transferred to poly(vinylidene difluoride)membranes. Proteins were stained with Coomassie blue, and protein bands were excised and then sequenced by automated Edman degradation (Applied Biosystems, Procise 492) by the Laboratory for Macromolecular Structure, Purdue University.

### Example 9

Generation of Peptide Antisera

Peptide antisera to the tNOX terminus containing the putative adenine binding site KQEMTGVGASLEKRW (SEQ ID NO: 16) were generated in rabbits using standard technology by Covance Research Products Inc. (Dever, Pa.). The antisera were diluted 1:300 before use.

### Example 10

Generation of Polyclonal Antisera

The recombinant truncated tNOX from the recombinant E. coli extract was precipitated with 20% ammonium sulfate and the solubilize proteins were resolved on a 12% SDS-PAGE and stained with Coomassie blue. The tNOX protein bands were excised and chopped into fine pieces. The protein then was mixed with 0.5 ml complete Freund's adjuvant and injected into two rabbits. Three boosts of antigen in incomplete Freund's adjuvant were given in three weeks interval. The antisera were diluted 1:300 before use.

### Example 11

RNA Isolation and Northern Analyses.

Total RNA was prepared from HeLa (or other cells) using the guanidinium method described by Ausubel et al. (1992), Current Protocols in Molecular Biology, Wiley Interscience, New York, N.Y. Denatured RNA was transferred to nitrocellulose membranes for hybridization and autoradiography essentially as described in Sambrook et al. [(1989) supra].

mRNA is isolated from biological samples, biopsy material, tumor tissue or the like and resolved using gel electrophoresis. Suitable conditions include 1.2% agarose and 2.2 moles/liter formaldehyde. mRNA sizes are estimated by comparison to marker molecules, such as the 0.28 to 6.58 kb markers available commercially, for example, from Promega, Madison, Wis. Lanes containing marker molecules are stained with ethidium bromide and photographed with UV illumination. Transfer of RNA molecules from the gel to nitrocellulose filters is accomplished as described by Maniatis et al. (1982) supra. Blots are prehybridized at 42C. for 2 h with 50% formamide, 5×SSPE, 2× Denhardt's solution, and 0.1% sodium dodecyl sulfate (SDS). Denatured radiolabeled or other labeled probe nucleic acid is added directly to the prehybridization fluid and the incubation is continuous for an additional 16-24 h. The blots are then washed for 20 min at room temperature in 1×SSC, 0.1% SDS, followed by three washes of 20 min each at 68C. in 0.2×SSC, 0.1% SDS. The labeled probe is then visualized according to the label used. Where the label is radioactive, autoradiography can be used.

Samples for use in nucleic acid-based diagnostic methods include 15-25 ml peripheral blood specimens For biopsy or other tumor tissue specimens, the tissue or biopsy sample is frozen in liquid nitrogen immediately after collection. Ground tissue or cells from blood are dissolved in guanidinium thiocyanate, left for 15 min at 50C. and then centrifuged at 3000 rpm at 5 min. The supernatant is layered over cesium chloride and centrifuged. The RNA pellet is dissolved in diethylpyrocarbonate. About 2 mg RNA are used for cDNA synthesis using commercially available reagents according to the supplier's instructions (e.g., Promega). PCR can be carried out using commercially available reagents and primers specific for the tNOX mRNA. The integrity of RNA samples is confirmed using an irrelevant gene product, for Example glyceraldehyde phosphate 3 dehydrogenase, the sequence of which is well known.

### Example 12

Mutagenic Oligonucleotides and Site-Directed Mutagenesis

Eight sets of oligonucleotides were designed to replace amino acid residues potentially involved in tNOX activity by site-directed mutagenesis according to Braman et al. (1996). Cysteine codons corresponding to C505, C510, C558, C569, C575, and C602, were replaced by alanine codons. The coding sequence was independently modified to replace a methionine of the putative drug binding site with an alanine (M396A). The tNOX coding sequence was independently modified to replace a glycine in the potential adenine binding site with a valine (G592V). Oligonucleotides were as follows: C505A: 5′-GAAAAGGAAAGCGCCGCTTCTAGGCTGTGTGCC-3′ (forward, SEQ ID NO: 17), 5′-GGCACACAGTCCCTAGAAGCGGCGCTTTCCTTTTC-3′ (reverse, SEQ ID NO: 18); C510A: 5′-GCTTCTAGGCTGGCCGCCTCAAACCAGGATAGCG-3′ (forward, SEQ ID NO: 19), 5′-CGCTATCCTGGTTTGAGGCGGCCAGCCTAGAAGC-3′ (reverse, SEQ ID NO: 20); C558A: 5′-GCAAGCATTGAATACATCGCTTCCTACTTGCACCGTCTTG-3′ (forward, SEQ ID NO: 21), 5′-CAAGACGGTGCAAGTAGGAAGCGATGTATTCAATGCTTGC-3′ (reverse, SEQ ID NO: 22); C569A: 5′-CGTCTTGATAATAAGATCGCCACCAGCGATGTGGAGTG-3′ (forward, SEQ ID NO: 23), 5′-CACTCCACATCGCTGGTGGCGATCTTATTATCAAGACG-3′ (reverse, SEQ ID NO: 24); C575A: 5′-CCAGCGATGTGGAGGCCCTCATGGGTAGACTCC-3′ (forward, SEQ ID NO: 25), 5′-GGAGTCTACCCATGAGGGCCTCCACATCGCTGG-3′ (reverse, SEQ ID NO: 26); C602A: 5′-GAAAAGAAGATGGAAATTCGCTGGCTTCGAGGGCTTGAAG-3′ (forward, SEQ ID NO: 27), 5′-CTTCAAGCCCTCGAAGCCAGCGAATTTCCATCTCTTTTC-3′ (reverse, SEQ ID NO: 28); M396A: 5′-GTCTGATGATGAAATAGAAGAAGCGACAGAAACAAAAGAAACTGAGG-3′ (forward, SEQ ID NO: 29), 5′-CCTCAGTTTCTTTTGTTTCTGTCGCTTCTTCTATTTCATCATCAGAC-3′ (reverse, SEQ ID NO: 30); G592V: 5′-CAGGAAATGACTGGAGTTGTGGCCAGCCTGGAAAAGAG-3′ (forward, SEQ ID NO: 31), 5′-CTCTTTTCCAGGCTGGCCACAACTCCAGTCATTTCCTG-3′ (reverse, SEQ ID NO: 32).

For the site-directed mutagenesis, the high fidelity thermostable Pfu DNA polymerase, low cycle number, and primers designed only to copy the parental strand in a linear fashion were used to minimize unwanted second site mutations. Double-stranded, super-coiled expression plasmid pET11tNOX (40 ng) and mutagenic sense and antisense primers (100 ng) were employed in a 50-μl reaction mixture containing deoxyribonucleotides, buffer, and Pfu DNA polymerase according to the manufacturer's protocol (Stratagene, La Jolla, Calif.). The cycling parameters were 95C. for 30 sec, 55C. for 1 min, and 68C. for 12.8 min for a total of 16 cycles. The linear amplification product was treated with endonuclease DpnI (10 units/μl) for 1 h to eliminate the parental template. Subsequently, an aliquot of 4 μl of this reaction mixture containing the double-nicked mutated plasmid was used for the transformation of supercompetent E. coli XL-1 Blue cells (Stratagene). All mutants were analyzed by DNA sequencing to confirm that the correct replacements were achieved.

References

Bird, C. (1999) Direct submission of human DNA sequence from clone 875H3 (part of APK1 antigen), GenBank Accession no. AL049733.

Braman et al. (1996) Meth. Mol. Biol. 57, 31-44.

Bridge et al. (2000) Biochim. Biophys. Acta 1463, 448-458.

Bruno, M. et al. (1992) Biochem J. 24:625-628.

Chang, K. and Pastan, I. (1994) Int. J. Cancer 57:90-97.

Chang, J. (2000) Biochem. Pharmacol. 59, 211-219.

Chang, K. and Pastan, I. (1994) Int. J. Cancer 57, 90-97.

Chang, K. and Pastan, I. (1996) Proc. Natl. Acad. Sci. USA 93, 136-140.

Chang et al. (1992) Cancer Res. 52, 181-186.

Chueh, P. J. et al. (1997) Arch. Biochem. Biophys. 342, 38-47.

Dai, S. et al. (1997) Mol. Cell. Biochem. 166:101-109.

DeHahn et al. (1997) Biochim. Biophys. Acta 1328, 99-108.

del Castillo-Olivares et al. (1998) Arch. Biochem. Biophys. 358, 125-140.

Duke, S. O. (1990) Environ. Health Perspectives 87, 263.

Freedman, R. B. (1989) Cell 57, 1069-1072.

Gilberger et al. (1997) J. Biol. Chem. 272, 29584-29589.

Gilberger et al. (1998) FEBS Lett. 425, 407-410.

Grabau, C. and Cronan, J. E. (1986) Nucleic Acids Res. 14, 5449.

Hanahan and Meselson (1980) Using Antibodies in Immunological Screening.

Kim et al. (1997) Biochim. Biophys. Acta 1324, 171-181.

Kishi et al. (1999) Biochim. Biophys. Acta 1412, 66-77.

Kliman, R. M. and Hey, J. (1993) Genetics 133, 375-387.

Kozak, M. (1987) Nucleic Acids Res. 15, 8125-8148.

Kyte, J. and Doolittle, R. F. (1982) J. Mol. Biol. 157, 105-132.

Leblanc et al. (1995) J. Mol. Biol. 250, 484-495.

MacBeth et al. (2000) Science 289, 938-941.

Morré, D. J. and Brightman, A. O. (1991) J. Bioenerg. and Biomem. 23:469-489.

Morré, D. J. et al. (1995a) Biochim. Biophys. Acta 1236:237-243.

Morré, D. J. et al. (1995b) Biochim. Biophys. Acta 1240:11-17.

Morré, D. J. et al. (1995c) Proc. Natl. Acad. Sci. USA 92:1831-1835.

Morré, D. J. (1995) Biochim. Biophys. Acta 1240, 201-208.

Morré, D. J. (1998) in Plasma Membrane Redox Systems and their role in Biological Stress and Disease. NADH oxidase: A multifunctional ectoprotein of the eukarotic cell surface. (Asard, E., Bérczi, A., and Caubergs, R. J., eds.) Kluwer Academic Publishers, Dordrecht, pp. 121-156.

Morré, D. J. (1998b) Mol. Cell. Biochem. 187, 41-46.

Morré, D. J., and Morré, D. M. (1995) J. Bioenerg. Biomembr. 27, 137-144.

Morré, D. J., and Reust, T. (1997) J. Bioenerg. Biomembr. 29, 281-289.

Morré et al. (1995b) Protoplasma 184, 203-208.

Morré et al. (1996a) Biochim. Biophys. Acta 1280, 197-206.

Morré et al. (1997a) Arch. Biochem. Biophys. 342, 224-230.

Morré et al. (1997b) J. Bioenerg. Biomemb. 29, 269-280.

Morré et al. (1997c) Biochim. Biophys. Acta 1325, 117-125.

Morré et al. (1998) J. Bioenerg Biomemb. 30, 477-487.

Morré et al. (1998a) Biochim. Biophys. Acta 1369, 185-192.

Morré et al. (1999) Mol. Cell. Biochem. 200, 7-13.

Morré et al. (2000) Biochem. Pharmacol. 60, 937-946.

Ohnishi et al. (1995) J. Biol. Chem. 270, 5812-5817.

Pogue et al. (2000) Biochim. Biophys. Acta 14662, 1-8.

Russel, M. and Model, P. (1988) J. Biol. Chem. 263, 9015-9019.

Schook, L. B. (1987) Immunology Series, Vol. 3, pp99-101. Marcel Dekker. Inc.

Sharrosh, B. S., and Dixon, R. A. (1991) Proc. Natl. Acad. Sci. USA 88, 10941-10945.

Shininá et al. (1996) Eur. J. Biochem. 237, 433-439.

Sugano et al. (2000) Direct submission of cDNA sequence to GenBank database. (Accession no. AK000353).

Sun et al. (2000) Biochim. Biophys. Acta 14665, 1-12.

Wang et al. (1998) FASEB J. 12, A519.

Wilkinson et al. (1996) Arch. Biochem. Biophys. 336, 275-282.

Yano et al. (1997) J. Biol. Chem. 272, 4201-4211.

TABLE 1

Nucleotide and deduced amino acid sequences of the tNOX-cDNA. The first
translation indicated is at nucleotides 23-25 (ATG) with termination at 1855-
1857 (TAA). Putative signal peptides are underlined and the signal peptide
cleavage site are indicated by arrows. The putative quinone binding sequence,
E394EMTE, is indicated by long dash-dot dot line. The copper binding site
H546VH and down stream H467 are shown by asterisks. The possible adenine
(NADH) binding sequence, T589GVGASL, is indicated by a dashed line.

```
      1
GTTCACAGTTGAGGACCACACAATGCAAAGAGATTTTAGATGGCTGTGGGTCTACGAAATAGGCTATGCAGCCGATAA
CAGTAGAACTCTG
      1 M Q R D F R W L W V Y E I G Y A A D N S R T L
    92
AACGTGGATTCCACTGCAATGACACTACCTATGTCTGATCCAACTGCATGGGCCACAGCAATGAATAATCTTGGAATG
GCACCGCTGGGA
    24 N V D S T A M T L P M S D P T A W A T A M N N L G M A P L G
  182
ATTGCCGGACAACCAATTTTACCTGACTTTGATCCTGCTCTTGGAATGATGACTGGAATTCCACCAATAACTCCAATG
ATGCCTGGTTTG
    54 I A G Q P I L P D F D P A L G M M T G I P P I T P M M P G L
  272
GGAATAGTACCTCCACCAATTCCTCCAGATATGCCAGTAGTAAAAGAGATCATACACTGTAAAAGCTGCACGCTCTTC
CCTCCAAATCCA
    84 G I V P P P I P P D M P V V K E I I H C K S C T L F P P N P
  362
AATCTCCCACCTCCTGCAACCCGAGAAAGACCACCAGGATGCAAAACAGTATTTGTGGGTGGTCTGCCTGAAAATGGG
ACAGAGCAAATC
  114 N L P P P A T R E R P P G C K T V F V G G L P E N G T E Q I
  452
ATTGTGGAAGTTTTCGAGCAGTGTGGAGAGATCATTGCCATTCGCAAGAGCAAGAAGAACTTCTGCCACATTCGCTTT
GCTGAGGAGTAC
  144 I V E V F E Q C G E I I A T R K S K K N F C H I R F A E E Y
  542
ATGGTGGACAAAGCCCTGTATCTGTCTGGTTACCGCATTCGCCTGGGCTCTAGTACTGACAAGAAGGACACAGGCAGA
CTCCACGTTGAT
  174 M V D K A L Y L S G Y R I R L G S S T D K K D T G R L H V D
  632
TTCGCACAGGCTCGAGATGACCTGTATGAGTGGGAGTGTAAACAGCGTATGCTAGCCAGAGAGGAGCGCCATCGTAGA
AGAATGGAAGAA
  204 F A Q A R D D L Y E W E C K Q R M L A R E E R H R R R M E E
  722
GAAAGATTGCGTCCACCATCTCCACCCCCAGTGGTCCACTATTCAGATCATGAATGCAGCATTGTTGCTGAAAAATTA
AAAGATGATTCC
  234 E R L R P P S P P P V V H Y S D H E C S I V A E K L K D D S
  812
AAATTCTCAGAAGCTGTACAGACCTTGCTTACCTGGATAGAGCGAGGAGAGGTCAACCGTCGTAGCGCCAATAACTTC
TACTCCATGATC
  264 K F S E A V Q T L L T W I E R G E V N R R S A N N F Y S M I
  902
CAGTCGGCCAACAGCCATGTCCGCCGCCTGGTGAACGAGAAAGCTGCCCATGAGAAAGATAGGAAGAAGCAAAGGAG
AAGTTCAAGCAG
  294 Q S A N S H V R R L V N E K A A H E K D M E E A K E K F K Q
  992
GCCCTTTCTGGAATTCTCATTCAATTTGAGCAGATAGTGGCTGTGTACCATTCCGCCTCCAAGCAGAAGGCATGGGAC
CACTTCACAAAA
  324 A L S G I L I Q F E Q I V A V Y H S A S K Q K A W D H F T K
1082
GCCCAGCGGAAGAACATCAGCGTGTGGTGCAAACAAGCTGAGGAAATTCGCAACATTCATAATCATGAATTAATGGGA
ATCAGGCGAGAA
  354 A Q R K N I S V W C K Q A E E I R N I H N D E L M G I R R E
1172
GAAGAAATGGAAATGTCTGATGATGAAATAGAAGAAATGACAGAAACAAAAGAAACTGAGGAATCAGCCTTAGTATCA
CAGGCAGAAGCT
  384 E E M E M S D D E I E E M T E T K E T E E S A L V S Q A E A
1262
CTGAAGGAAGAAAATGACAGCCTCCGTTGGCAGCTCGATGCCTACCGGAATGAAGTAGAACTGCTCAAGCAAGAACAA
GGCAAAGTCCAC
  414 L K E E N D S L R W Q L D A Y R N E V E L L K Q E Q G K V H
1352
AGAGAAGATGACCCTAACAAAGAACAGCAGCTGAAACTCCTGCAACAAGCCCTGCAAGGAATGCAACAGCATCTACTC
AAAGTCCAAGAG
  444 R E D D P N K E Q Q L K L L Q Q A L Q G M Q Q H L L K V Q E
1442
GAATACAAAAAGAAAGAAGCTGAACTTGAAAAACTCAAAGATGACAAGTTACAGGTGGAAAAAATGTTGGAAAATCTT
AAAGAAAAGGAA
  474 E Y K K K E A E L E K L K D D K L Q V E K M L E N L K E K E
1532
AGCTGTGCTTCTAGGCTGTGTGCCTCAAACCAGGATAGCGAATACCCTCTTGAGAAGACCATGAACAGCAGTCCTATC
AAATCTGAACGT
  504 S C A S R L C A S N Q D S E Y P L E K T M N S S P I K S E R
1622
GAAGCACTGCTAGTGGGGATTATCTCCACATTCCTTCATGTTCACCCATTTGGAGCAAGCATTGAATACATCTGTTCC
TACTTGCACCGT
  534 E A L L V G I I S T F L H* V* H* P F G A S I E Y I C S Y L H R
1712
CTTGATAATAAGATCTGCACCAGCGATGTGGAGTGTCTCATGGGTAGACTCCAGCATACCTTCAAGCAGGAAATGACT
GGAGTTGGAGCC
  564 L D N K I C T S D V E C L M G R L Q H T F K Q E M T G V G A
1802
AGCCTGGAAAAGAGATGGAAATTCTGTGGCTTCGAGGGCTTGAAGCTGACCTAAATCTCTTTGCCTAACAACTTGGGA
TCCTGAAGATAA
  594 S L E K R W K F C G F E G L K L T Stop
1892
ATATGTGTTGGACAAGCATAGAAAGTGATTTATATTTTTAATGGTTTTCAAGTGGAAGTTCCTTTGAATTTGTCAGTT
CATTCCTGGAAA
1982
ATCTTTTGAGTTAAAATAAGGATCCTAGGACAGCACCTCGAACTACAGGCCCTAAAGAGAAATTGCCTCAAACCACAA
GTGCTGTAACTT
2072
CCTCCCCTTTCTGTCAATTGGTTGTCTTTAAATATTGCAAAAGTCCTGATGCTAAACAGTATTTGGAGTGTTTTCAGT
GTCTGTACTACT
2162
GTTGTACACCTTGGTATTTTTTTAAACACTGTTAACTGAAATGTTTTGATGATTTTATGTGATTTGTGTTTCTAAACT
TCTCTTTACATT
2252
AATGTTGTTACTGGTGAAAGGCATGAGAGCAGCACTAAGTCCTCTGTGTAACTGCCATTGTCTTTCCAATCCCCAGTA
GACCAGTAAATA
2342
AATAACACATCAGTGTCTTCTAGAAGGTGCCTGACCAGGTTCACCTTTTAAACGACAAAGCATGGTTTGTGGCTTTTT
GCAAAATTACTA
2432
TGAACCAAAAGTTGACAAATGTTCCAAAGTTATTTTCTCTAACATATCACATTAAAGATCTGTTTCAGAATTGTAAAA
AGTACATCTAGA
2522
TGTGTTTACAGAAAGCAAGTATCCAGTATGACTGGCATGTGTTCATGCTATTCAGAATCACTTGTAAATAGTCTGCTT
TTAAAGGAGGGC
2612
ATGTTCAGTTTTCTGTGAATTAAAATATGCTCATGTGTGGGCACACACGCACAAACACACACACGCACGCACACAGTG
GCAGAAGGGATT
2702
TATATTAATATTCTTTCCCCTCTGGCCTTCTTACAGTCTGTTGGTCCCTTTGCTTCTGTTGTCAGTGTGTTGAATTGC
AAACCGAGTACT
2792
GCTGTAAATACTATGTTTACTTCATGCTGAATGTTTGCAAAGACTTGATATAAGTATTAATAGTAATGAATCAATGAA
TAAATAATGAGC
2882
TAGGGTTTGTGAGGCTTTCTACAAATAGGTCAGCTCCACCTGGAGTGCGAATTGCCAGAGACACCTTGGTAGTGCCCA
TCGGCAAATCGC
2972
AATGGCAGCATGTGAGTGGACCATTCAGAAACTTCTGCTTGGTGGAAAGTAAACAGAGAGGATGGAGGTTTGGGGCGA
ATGTCCTGAGGC
3062
AGAGATGGTCTTTATTGTGTGTGGTGGTGGTTGTGGTATTTATAATAATGCAAGCATACCCTCCCTTGAGTCTCAATT
GAAGATAAAAGA
3152
ATGTACTGAGCAAGCAAAGCCAATGGAGAGTATTTCACAAAAATACTTTGTAAATGAGATGCCAGTAGTGTTCAAAGT
TGTATTTTTAAA
3242
AGATAAATATTCCTTTTTATACCTCAGTTTTGTGTCCTGTTTTTTAATGACTTACGCTCTAAGTAATCCATTAGTAGT
TATCTCAGTCCC
3332
TCCCTTTGGGTTACTAGAATGTTGGAAAAAGATGCCAACTCTGTCTTGACAACTGGAAACACGGTTCCACAGCAGCCC
ATTCGTGCTGAA
3422
AACTGGCTTCCCCCCTCAAGCACCCTGCTGTGGCACCAGCAGGAACCTCACGTTAATTTTACACTAGCTTGCTCACTG
ATCCATCTCTCA
3512
TCAATGCTACGGAAGGCTTTGATTCATCAGTCTCGGGCTCTTGGAATACCTAATTTTAATAATATCTATGAAATCAAG
GGAAACTTTCCA
3602
TTTACAGTTATTTCTTCTTTAAATAAACTAAATTAATTTTTAGGGGAGAGCACTAGCAAAAAGAGCTAATGCATGCGG
GGTTTAATACCT
3692
AGGTGATGGGTTGAGGTGCAGCAAAACCACCATGGCACACGTTCACCTATGTAACAAACCTGCACATCCTGCACATGT
ACCCCGGAACTT
3782 ACTTAAAA
```

TABLE 2
Comparison of amino acid sequences within the known quinone
and sulfonylurea binding sites of several proteins.

| PROTEIN | SEQUENCE | SEQUENCE ID NO. |
|---|---|---|
| Q_{B}-protein⁺ | S A M H G | 33 |
| L/M-subunit⁺ | L A M H G | 34 |
| Acetolactate synthetase | L G M G H | 35 |
| (Tobacco)^{+*} | | |
| Pyruvate oxidase⁺ | A T M H W | 36 |
| Preliminary consensus | X A M H G | 37 |
| D₁-Synechococcus | E T M R F | 38 |
| NADH (ubiquinone) dehydrdo- | G E M R B | 39 |
| genase | | |
| Bovine serum albumin | B T M R E | 40 |
| Human serum albumin | A T L R E | 41 |
| Acetolactate synthetase (Brassica) | B T L R E | 42 |

⁺Binds quinone
*Binds sulfonylurea

TABLE 3
Effect of site-directed mutagenesis of ttNOX on NADH oxidase
enzyme activity, period length and inhibition by capsaicin.

| | Enzymatic | Period | Complete inhibition |
|---|---|---|---|
| Mutation⁺ | activity | length | by 1 μM capsaicin |
| None | + | 23 min | + |
| C505A | | | |
| C510A | + | 39 min | + |
| C558A | + | 39 min | + |
| C569A | | | |
| C575A | + | 36 min | + |
| C602A | 36 min | + | |
| M396A | + | 23 min | − |
| G502A | − | | |

⁺Resequencing confirmed the expected DNA sequences for each of the indicated amino acid replacements.

TABLE 4
Response of COS cells stably transfected with tNOX cDNA to
targeted drugs.

| | EC₅₀ | |
|---|---|---|
| Drug | Nontransfected | tNOX |
| Capsaicin | 13 | 1.3 |
| EGCg | 10 | 0.1 |
| Adriamycin | 0.3 | 0.04 |
| LY181984 (active) | 20 | 3 |
| LY1819845 (inactive) | >100 | >100 |
| Methotrexate | 1 | 1 |

Capsaicin, 8-methyl-N-vanillyl-6-noncamide
EGCg, epigallocatechin gallate
LY181984, N-(4-methylphenylsulfonyl-N')-4-chlorophenylurea
LY181985, N-(4-methylphenylsulfonyl-N')-4-phenylurea

We claim:

1. A non-naturally occurring recombinant DNA molecule comprising a portion encoding an NADH oxidase/protein disulfide-thiol interchange polypeptide, said portion consisting essentially of a nucleotide sequence selected from the group consisting of SEQ ID NO: 1, nucleotides 23 to 1852; SEQ ID NO: 1, nucleotides 680 to 1852; and a sequence which hybridizes under stringent conditions to one of the foregoing sequences and wherein said hybridizing sequence encodes a neoplastic marker protein of the cell surface having NADH oxidase/protein disulfide-thiol interchange activity.

2. The non-naturally occurring recombinant DNA molecule of claim 1 wherein said polypeptide consists essentially of an amino acid sequence selected from the group consisting of SEQ ID NO: 2, amino acids 1 to 610 and SEQ ID NO: 2, amino acids 220 to 610.

3. The non-naturally occurring recombinant DNA molecule of claim 2 wherein portion encoding said polypeptide consists essentially of a nucleotide sequence encoding said NADH oxidase/protein disulfide-thiol interchange polypeptide as given in SEQ ID NO: 1, nucleotides 23 to 1852 (exclusive of a translation termination codon).

4. The non-naturally occurring recombinant DNA molecule of claim 2 wherein portion encoding said polypeptide consists essentially of a nucleotide sequence encoding said NADH oxidase/protein disulfide-thiol interchange polypeptide as given in SEQ ID NO: 1, nucleotides 680 to 1852 (exclusive of a translation termination codon).

5. The non-naturally occurring recombinant DNA molecule of claim 3 further comprising a translation termination codon, wherein said translation termination codon is TGA, TAA or TAG and it is immediately downstream of nucleotide 1852 of SEQ ID NO: 1.

6. The non-naturally occurring recombinant DNA molecule of claim 4 further comprising a translation termination codon, wherein said translation termination codon is TGA, TAA or TAG and it is immediately downstream of nucleotide 1852 of SEQ ID NO: 1.

7. A host cell transformed or transfected to contain the recombinant DNA molecule of claim 1.

8. The host cell of claim 7 which is a bacterial cell.

9. The host cell of claim 8 wherein said bacterial cell is an Escherichia coli cell.

10. The host cell of claim 7 wherein said cell is a eukaryotic cell.

11. The host cell of claim 10 wherein said cell is a mammalian cell.

12. The host cell of claim 11 wherein said cell is a COS cell.

13. The host cell transformed or transfected to contain the recombinant DNA molecule of claim 2.

14. The host cell of claim 13 which is a bacterial cell.

15. The host cell of claim 14 wherein said bacterial cell is an Escherichia coli cell.

16. The host cell of claim 13 wherein said cell is a eukaryotic cell.

17. The host cell of claim 16 wherein said cell is a mammalian cell.

18. The host cell of claim 17 wherein said cell is a COS cell.

19. A method for recombinantly producing a NADH oxidase/protein disulfide-thiol interchange active polypeptide in a host cell, said method comprising the steps of:
a) infecting or transforming a host cell with a vector comprising a promoter active in said host cell, said promoter being operably linked to a coding region for said NADH o.xidase-protein disulfide-thiol interchange polypeptide having an amino acid sequence selected from the group consisting of SEQ ID NO: 2, amino acids 1 to 610; SEQ ID NO: 2, amino acids 220 to 610, or a coding sequence encoding a NADH oxidase-protein disulfide-thiol interchange polypeptide hybridizing under stringent conditions to a nucleic acid molecule as given in SEQ ID NO: 1, nucleotides 23 to 1852 or nucleotides 680 to 1852, to produce a recombinant host cell; and
b) culturing the recombinant host cell under conditions wherein said polypeptide is xpressed.

20. A method for determining neoplasia in a mammal, said method comprising the steps of:
a) detecting the presence, in a biological sample from a mammal, of a ribonucleic acid molecule encoding a NADH oxidase/protein disulfide thiol interchange protein associated with neoplastic cells as compared to a ribonucleic acid molecule encoding a NADH oxidase associated with normal,cells, wherein the step of detecting is carried out using hybridization under stringent conditions or using a polymerase chain reaction in which a perfect match of primer to template is required, where a hybridization probe or primer consists essentially consists essentially of at least 15 consecutive nucleotides of a nucleotide sequence as given in SEQ ID NO: 1;
b) correlating the result obtained with said sample in step (a), where the presence of the ribonucleic acid molecule in the biological sample is indicative of the presence of neoplasia.

21. The method of claim 20 wherein the hybridization probe consists essentially of a nucleotide sequence as given in SEQ ID NO: 1, nucleotides 680-1652.

22. The method of claim 20 wherein the hybridization probe or primer consists essentially of a nucleotide sequence as given in SEQ ID NO: 1, nucleotides 23 to 1852.

23. An antibody preparation which specifically binds to an antibody selected from the group consisting of a protein characterized by an amino acid sequence as given in SEQ ID NO: 2, amino acids 1-610, a protein characterized by an amino acid sequence as given in SEQ ID NO: 2, amino acids 220-610 or a protein characterized by an amino acid sequence as given in SEQ ID NO: 16.

24. A method for determining neoplasia in a mammal, said method comprising the steps of:
a) detecting the presence, in a biological sample from a mammal, of a NADH oxidase/protein disulfide thiol interchange protein associated with neoplastic cells as compared to normal cells, wherein the step of detecting is carried out using an antibody specific for a protein characterized by an amino acid sequence as given in SEQ ID NO: 2, amino acids 1-610, a protein characterized by an amino acid sequence as given in SEQ ID NO: 2, amino acids 220-610 or a protein characterized by an amino acid sequence as given in SEQ ID NO: 16; and
b) correlating the result obtained with said sample in step (a), where the presence of the protein in the biological sample is indicative of the presence of neoplasia.


SEQUENCE LISTING (42 sequences)

SEQ ID NO: 1 - DNA, 3789 bp, Homo sapiens
  CDS (23)..(1852)
gttcacagttgaggaccacacaatgcaaagagattttagatggctgtgggtctacgaaataggctatgcagccgataacagtagaactctgaacgtggattccactgcaatgacactacctatgtctgatccaactgcatgggccacagcaatgaataatcttggaatggcaccgctgggaattgccggacaaccaattttacctgactttgatcctgctcttggaatgatgactggaattccaccaataactccaatgatgcctggtttgggaatagtacctccaccaattcctccagatatgccagtagtaaaagagatcatacactgtaaaagctgcacgctcttccctccaaatccaaatctcccacctcctgcaacccgagaaagaccaccaggatgcaaaacagtatttgtgggtggtctgcctgaaaatgggacagagcaaatcattgtggaagttttcgagcagtgtggagagatcattgccattcgcaagagcaagaagaacttctgccacattcgctttgctgaggagtacatggtggacaaagccctgtatctgtctggttaccgcattcgcctgggctctagtactgacaagaaggacacaggcagactccacgttgatttcgcacaggctcgagatgacctgtatgagtgggagtgtaaacagcgtatgctagccagagaggagcgccatcgtagaagaatggaagaagaaagattgcgtccaccatctccacccccagtggtccactattcagatcatgaatgcagcattgttgctgaaaaattaaaagatgattccaaattctcagaagctgtacagaccttgcttacctggatagagcgaggagaggtcaaccgtcgtagcgccaataacttctactccatgatccagtcggccaacagccatgtccgccgcctggtgaacgagaaagctgcccatgagaaagatatggaagaagcaaaggagaagttcaagcaggccctttctggaattctcattcaatttgagcagatagtggctgtgtaccattccgcctccaagcagaaggcatgggaccacttcacaaaagcccagcggaagaacatcagcgtgtggtgcaaacaagctgaggaaattcgcaacattcataatgatgaattaatgggaatcaggcgagaagaagaaatggaaatgtctgatgatgaaatagaagaaatgacagaaacaaaagaaactgaggaatcagccttagtatcacaggcagaagctctgaaggaagaaaatgacagcctccgttggcagctcgatgcctaccggaatgaagtagaactgctcaagcaagaacaaggcaaagtccacagagaagatgaccctaacaaagaacagcagctgaaactcctgcaacaagccctgcaaggaatgcaacagcatctactcaaagtccaagaggaatacaaaaagaaagaagctgaacttgaaaaactcaaagatgacaagttacaggtggaaaaaatgttggaaaatcttaaagaaaaggaaagctgtgcttctaggctgtgtgcctcaaaccaggatagcgaataccctcttgagaagaccatgaacagcagtcctatcaaatctgaacgtgaagcactgctagtggggattatctccacattccttcatgttcacccatttggagcaagcattgaatacatctgttcctacttgcaccgtcttgataataagatctgcaccagcgatgtggagtgtctcatgggtagactccagcataccttcaagcaggaaatgactggagttggagccagcctggaaaagagatggaaattctgtggcttcgagggcttgaagctgacctaaatctctttgcctaacaacttgggatcctgaagataaatatgtgttggacaagcatagaaagtgatttatatttttaatggttttcaagtggaagttcctttgaatttgtcagttcattcctggaaaatcttttgagttaaaataaggatcctaggacagcacctcgaactacaggccctaaagagaaattgcctcaaaccacaagtgctgtaacttcctcccctttctgtcaattggttgtctttaaatattgcaaaagtcctgatgctaaacagtatttggagtgttttcagtgtctgtactactgttgtacaccttggtatttttttaaacactgttaactgaaatgttttgatgattttatgtgatttgtgtttctaaacttctctttacattaatgttgttactggtgaaaggcatgagagcagcactaagtcctctgtgtaactgccattgtctttccaatccccagtagaccagtaaataaataacacatcagtgtcttctagaaggtgcctgaccaggttcaccttttaaacgacaaagcatggtttgtggctttttgcaaaattactatgaaccaaaagttgacaaatgttccaaagttattttctctaacatatcacattaaagatctgtttcagaattgtaaaaagtacatctagatgtgtttacagaaagcaagtatccagtatgactggcatgtgttcatgctattcagaatcacttgtaaatagtctgcttttaaaggagggcatgttcagttttctgtgaattaaaatatgctcatgtgtgggcacacacgcacaaacacacacacgcacgcacacagtggcagaagggatttatattaatattctttcccctctggccttcttacagtctgttggtccctttgcttctgttgtcagtgtgttgaattgcaaaccgagtactgctgtaaatactatgtttacttcatgctgaatgtttgcaaagacttgatataagtattaatagtaatgaatcaatgaataaataatgagctagggtttgtgaggctttctacaaataggtcagctccacctggagtgcgaattgccagagacaccttggtagtgcccatcggcaaatcgcaatggcagcatgtgagtggaccattcagaaacttctgcttggtggaaagtaaacagagaggatggaggtttggggcgaatgtcctgaggcagagatggtctttattgtgtgtggtggtggttgtggtatttataataatgcaagcataccctcccttgagtctcaattgaagataaaagaatgtactgagcaagcaaagccaatggagagtatttcacaaaaatactttgtaaatgagatgccagtagtgttcaaagttgtatttttaaaagataaatattcctttttatacctcagttttgtgtcctgttttttaatgacttacgctctaagtaatccattagtagttatctcagtccctccctttgggttactagaatgttggaaaaagatgccaagtctgtcttgacaactggaaacagggttccacagcagcccattcgtgctgaaaactggcttcccccctgaagcaccctgctgtggcaccagcaggaagctcaggttaattttacactagcttgctcactgatgcatctctcatcaatgctacggaaggctttgattcatcagtctcgggctcttggaatacctaattttaataatatctatgaaatcaagggaaactttccatttacagttatttcttgtttaaataaactaaattaatttttaggggagagcagtaggaaaaagagctaatgcatgcggggtttaatacctaggtgatgggttgaggtgcagcaaaaccaccatggcacacgttcacctatgtaacaaacctgcacatcctgcacatgtaccccggaacttacttaaaa

SEQ ID NO: 2 - protein, 610 aa, Homo sapiens
MQRDFRWLWVYEIGYAADNSRTLNVDSTAMTLPMSDPTAWATAMNNLGMAPLGIAGQPILPDFDPALGMMTGIPPITPMMPGLGIVPPPIPPDMPVVKEIIHCKSCTLFPPNPNLPPPATRERPPGCKTVFVGGLPENGTEQIIVEVFEQCGEIIAIRKSKKNFCHIRFAEEYMVDKALYLSGYRIRLGSSTDKKDTGRLHVDFAQARDDLYEWECKQRMLAREERHRRRMEEERLRPPSPPPVVHYSDHECSIVAEKLKDDSKFSEAVQTLLTWIERGEVNRRSANNFYSMIQSANSHVRRLVNEKAAHEKDMEEAKEKFKQALSGILIQFEQIVAVYHSASKQKAWDHFTKAQRKNISVWCKQAEEIRNIHNDELMGIRREEEMEMSDDEIEEMTETKETEESALVSQAEALKEENDSLRWQLDAYRNEVELLKQEQGKVHREDDPNKEQQLKLLQQALQGMQQHLLKVQEEYKKKEAELEKLKDDKLQVEKMLENLKEKESCASRLCASNQDSEYPLEKTMNSSPIKSEREALLVGIISTFLHVHPFGASIEYICSYLHRLDNKICTSDVECLMGRLQHTFKQEMTGVGASLEKRWKFCGFEGLKLT

SEQ ID NO: 3 - protein, 8 aa, Artificial Sequence
  Description of Artificial Sequencepartial sequence of Chondous crispus mitochondrial ATP synthase protein 9.
TGVGAGVG

SEQ ID NO: 4 - protein, 4 aa, Artificial Sequence
  Description of Artificial Sequencepartial amino acid sequence surrounding sulfonylurea and quinone binding site in photosystem II.
AMHG

SEQ ID NO: 5 - protein, 5 aa, Artificial Sequence
  Description of Artificial Sequencepartial amino acid sequence of Synechococcus D1 protein.
ETMRE

SEQ ID NO: 6 - DNA, 27 bp, Artificial Sequence
  Description of Artificial Sequence oligonucleotide useful as primer.
gagtgtaaacagcatatgctagccaga

SEQ ID NO: 7 - DNA, 24 bp, Artificial Sequence
  Description of Artificial Sequence oligonucleotide useful as primer, for example.
tttctatgcttgtccaacacatat

SEQ ID NO: 8 - DNA, 30 bp, Artificial Sequence
  Description of Artificial Sequence oligonucleotide useful as primer, for example.
ggagatatacatatgggaattctcattcaa

SEQ ID NO: 9 - DNA, 24 bp, Artificial Sequence
  Description of Artificial Sequence oligonucleotide useful as primer, for example.
tttctatgcttgtccaacacatat

SEQ ID NO: 10 - DNA, 51 bp, Artificial Sequence
  Description of Artificial Sequence oligonucleotide useful as primer, for example.
gatatacatatgcatcatcatcatcatcatctagccagagaggagcgccat

SEQ ID NO: 11 - DNA, 24 bp, Artificial Sequence
  Description of Artificial Sequence oligonucleotide useful as primer, for example.
tttctatgcttgtccaacacatat

SEQ ID NO: 12 - DNA, 21 bp, Artificial Sequence
  Description of Artificial Sequence oligonucleotide useful as primer, for example.
tgggagtgtaaacagcgtatg

SEQ ID NO: 13 - DNA, 24 bp, Artificial Sequence
  Description of Artificial Sequence oligonucleotide useful as primer, for example.
tttctatgcttgtccaacacatat

SEQ ID NO: 14 - DNA, 21 bp, Artificial Sequence
  Description of Artificial Sequence oligonucleotide useful as primer, for example.
aaacttaagctttgggagtgt

SEQ ID NO: 15 - DNA, 24 bp, Artificial Sequence
  Description of Artificial Sequence oligonucleotide useful as primer, for example.
tttctatgcttgtccaacacatat

SEQ ID NO: 16 - protein, 15 aa, Artificial Sequence
  Description of Artificial Sequence  peptide sequence useful as antigen
KQEMTGVGASLEKRW

SEQ ID NO: 17 - DNA, 33 bp, Artificial Sequence
  Description of Artificial Sequence oligonucleotide useful as primer, for example.
gaaaaggaaagcgccgcttctaggctgtgtgcc

SEQ ID NO: 18 - DNA, 35 bp, Artificial Sequence
  Description of Artificial Sequence oligonucleotide useful as primer, for example.
ggcacacagtccctagaagcggcgctttccttttc

SEQ ID NO: 19 - DNA, 34 bp, Artificial Sequence
  Description of Artificial Sequence oligonucleotide useful as primer, for example.
gcttctaggctggccgcctcaaaccaggatagcg

SEQ ID NO: 20 - DNA, 34 bp, Artificial Sequence
  Description of Artificial Sequence oligonucleotide useful as primer, for example.
cgctatcctggtttgaggcggccagcctagaagc

SEQ ID NO: 21 - DNA, 40 bp, Artificial Sequence
  Description of Artificial Sequence oligonucleotide useful as primer, for example.
gcaagcattgaatacatcgcttcctacttgcaccgtcttg

SEQ ID NO: 22 - DNA, 40 bp, Artificial Sequence
  Description of Artificial Sequence oligonucleotide useful as primer, for example.
caagacggtgcaagtaggaagcgatgtattcaatgcttgc

SEQ ID NO: 23 - DNA, 38 bp, Artificial Sequence
  Description of Artificial Sequence oligonucleotide useful as primer, for example.
cgtcttgataataagatcgccaccagcgatgtggagtg

SEQ ID NO: 24 - DNA, 38 bp, Artificial Sequence
  Description of Artificial Sequence oligonucleotide useful as primer, for example.
cactccacatcgctggtggcgatcttattatcaagacg

SEQ ID NO: 25 - DNA, 33 bp, Artificial Sequence
  Description of Artificial Sequence oligonucleotide useful as primer, for example.
ccagcgatgtggaggccctcatgggtagactcc

SEQ ID NO: 26 - DNA, 33 bp, Artificial Sequence
  Description of Artificial Sequence oligonucleotide useful as primer, for example.
ggagtctacccatgagggcctccacatcgctgg

SEQ ID NO: 27 - DNA, 40 bp, Artificial Sequence
  Description of Artificial Sequence oligonucleotide useful as primer, for example.
gaaaagaagatggaaattcgctggcttcgagggcttgaag

SEQ ID NO: 28 - DNA, 39 bp, Artificial Sequence
  Description of Artificial Sequence oligonucleotide useful as primer, for example.
cttcaagccctcgaagccagcgaatttccatctcttttc

SEQ ID NO: 29 - DNA, 47 bp, Artificial Sequence
  Description of Artificial Sequence oligonucleotide useful as primer, for example.
gtctgatgatgaaatagaagaagcgacagaaacaaaagaaactgagg

SEQ ID NO: 30 - DNA, 47 bp, Artificial Sequence
  Description of Artificial Sequence oligonucleotide useful as primer, for example.
cctcagtttcttttgtttctgtcgcttcttctatttcatcatcagac

SEQ ID NO: 31 - DNA, 38 bp, Artificial Sequence
  Description of Artificial Sequence oligonucleotide useful as primer, for example.
caggaaatgactggagttgtggccagcctggaaaagag

SEQ ID NO: 32 - DNA, 38 bp, Artificial Sequence
  Description of Artificial Sequence oligonucleotide useful as primer, for example.
ctcttttccaggctggccacaactccagtcatttcctg

SEQ ID NO: 33 - protein, 5 aa, Artificial Sequence
  Description of Artificial Sequencequinone or sulfonylurea binding site of Qb protein
SAMHG

SEQ ID NO: 34 - protein, 5 aa, Artificial Sequence
  Description of Artificial Sequence  quinone or sulfonylurea binding site of L/M subunit
LAMHG

SEQ ID NO: 35 - protein, 5 aa, Artificial Sequence
  Description of Artificial Sequence  quinone or sulfonyurea binding site of acetolactate synthetase (tobacco)
LGMHG

SEQ ID NO: 36 - protein, 5 aa, Artificial Sequence
  Description of Artificial Sequence quinone or sulfonylurea binding site of pyruvate oxidase
ATMHW

SEQ ID NO: 37 - protein, 5 aa, Artificial Sequence
  Description of Artificial Sequencepreliminary consensus for quinone or sulfonylurea binding sites
XAMHG

SEQ ID NO: 38 - protein, 5 aa, Artificial Sequence
  Description of Artificial Sequence quinone or sulfonylurea binding site of D1 of Synechococcus
ETMRF

SEQ ID NO: 39 - protein, 5 aa, Artificial Sequence
  Description of Artificial Sequence quinone or sulfonylurea binding site of NADH (ubiquinone) dehydrogenase
GEMRE

SEQ ID NO: 40 - protein, 5 aa, Artificial Sequence
  Description of Artificial Sequence quinone or sulfonylurea binding site of bovine serum albumin
ETMRE

SEQ ID NO: 41 - protein, 5 aa, Artificial Sequence
  Description of Artificial Sequence quinone or sulfonylurea binding site of human serum albumin
ATLRE

SEQ ID NO: 42 - protein, 5 aa, Artificial Sequence
  Description of Artificial Sequence quinone or sulfonylurea binding site of acetolactate synthetase  (Brassica)
EDLRE